(19) United States
(12) Patent Application Publication
(10) Pub. No.: US 2018/0328884 A1
(43) Pub. Date: Nov. 15, 2018

(54) INTERFACES FOR SYRINGE-INJECTABLE ELECTRONICS

(71) Applicant: President and Fellows of Harvard College, Cambridge, MA (US)

(72) Inventors: Charles M. Lieber, Lexington, MA (US); Thomas Gerard Schuhmann, Cambridge, MA (US); Jun Yao, Northampton, MA (US); Guosong Hong, Cambridge, MA (US); Tian-Ming Fu, Ashburn, VA (US); Jungmin Lee, Seoul (KR); Hong-Gyu Park, Seoul (KR)

(73) Assignee: President and Fellows of Harvard College, Cambridge, MA (US)

(21) Appl. No.: 15/977,710

(22) Filed: May 11, 2018

Related U.S. Application Data
(60) Provisional application No. 62/505,562, filed on May 12, 2017.

(51) Int. Cl. G01N 27/414; A61B 5/04; A61B 5/00; A61B 5/0478; A61N 1/08

(52) U.S. Cl. CPC G01N 27/4146; A61B 5/04001; A61N 1/05; A61B 5/0478; A61N 1/08; A61B 5/6868

(57) ABSTRACT

The present invention generally relates to injectable electronics. In some aspects, the present invention is generally directed to systems and methods for interfacing an electrical cable with electrical elements, such as nanoscale wires, that are injected or otherwise introduced into a subject. The subject may be living or non-living. In one set of embodiments, electrical elements introduced within a subject may be placed in electrical communication to a circuit board using a plurality of electrically isolated contacts that the circuit board can clamp or otherwise connect to. The electrical contacts may be in electrical communication with the electrical elements using a joining portion. The circuit board can also be connected to an electrical cable that can be attached, for example, to a computer. In some cases, the electrical cable can be attached or detached to or from the circuit board, e.g., without requiring additional surgeries or interventions into the subject, to allow electrical communication with the electrical elements. Certain embodiments of the invention are also generated to systems and methods of making or using such devices, systems and methods of inserting such devices into a subject, kits including such device, or the like.

### RELATED APPLICATIONS

This application claims the benefit of U.S. Provisional Patent Application Ser. No. 62/505,562, filed May 12, 2017, entitled “Interfaces for Syringe-Injectable Electronics,” by Lieber, et al., incorporated herein by reference in its entirety.

### GOVERNMENT FUNDING

This invention was made with government support under Grant No. FA9550-14-1-0136 awarded by the U.S. Air Force, Office of Scientific Research. The government has certain rights in the invention.

### FIELD

The present invention generally relates to injectable electronics.

### BACKGROUND

Recent efforts in coupling electronics and tissues have focused on flexible, stretchable planar arrays that conform to tissue surfaces, or implantable microfabricated probes. Syringe-injectable mesh electronics with tissue-like mechanical properties and open macroporous structures is an emerging paradigm for mapping and modulating brain activity. Flexible macroporous structures have exhibited minimal non-invasiveness or the promotion of attractive interactions with neurons. These same structural features also pose challenges for precise targeted delivery in specific brain regions and quantitative input/output (I/O) connectivity needed for reliable electrical measurements. Accordingly, improvements for injectable electronics are needed.

### SUMMARY

The present invention generally relates to injectable electronics. The subject matter of the present invention involves, in some cases, interrelated products, alternative solutions to a particular problem, and/or a plurality of different uses of one or more systems and/or articles.

In one aspect, the present invention is generally directed to a device. In one set of embodiments, the device comprises a first portion comprising a plurality of electrical elements, a second portion comprising a plurality of electrically isolated contacts, and a joining portion electrically connecting the first portion and the second portion.

The present invention, in another aspect, is generally directed to a tube comprising a device for insertion into a subject. In one set of embodiments, the device comprises a first portion comprising a plurality of electrical elements, a second portion comprising a plurality of electrical contacts, and a joining portion connecting the first portion and the second portion. In some cases, at least some of the plurality of electrical contacts are curled around the joining portion within the tube.

In yet another aspect, the present invention is generally directed to a method. In one set of embodiments, the method comprises inserting at least a portion of a device comprising one or more electrical elements into a subject, and attaching the device to a circuit board.

The method, in another set of embodiments, includes providing a subject having a device inserted therein, where the device comprises one or more electrical elements in electrical communication with a plurality of electrically isolated contacts in electrical communication with a circuit board. In some cases, the method also includes repeatedly attaching and detaching an electrical cable to the circuit board.

In another aspect, the present invention encompasses methods of making one or more of the embodiments described herein, for example, an injectable device as discussed herein. In still another aspect, the present invention encompasses methods of using one or more of the embodiments described herein, for example, an injectable device as discussed herein.

Other advantages and novel features of the present invention will become apparent from the following detailed description of various non-limiting embodiments of the invention when considered in conjunction with the accompanying figures.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1B illustrate a device in accordance with one embodiment of the invention;

FIGS. 2A-2E illustrate a device with contacts, in another embodiment of the invention;

FIGS. 3A-3D illustrate electrical characteristics of certain devices, in other embodiments of the invention;

FIGS. 4A-4H illustrate a mouse having a device, in still another embodiment of the invention;

FIGS. 5A-5C illustrate a printed circuit board, in yet another embodiment of the invention;

FIG. 6 shows geometry for an I/O pad, in accordance with one embodiment of the invention;

FIGS. 7A-7B shows four-point probe measurements, in another embodiment of the invention;

FIG. 8 schematically illustrates a cross-section of one embodiment of the invention;

FIGS. 9A-9C illustrate a connection to a flat flexible cable (FFC), in another embodiment of the invention;

FIGS. 10-10D illustrates sizing I/O to reduce the possibility of short circuits, in yet another embodiment of the invention;

FIGS. 11A-11E illustrate a method of fabricating double-sided electrical contacts, in still another embodiment of the invention; and

FIGS. 12A-12M illustrate another method of fabricating double-sided electrical contacts, in yet another embodiment of the invention.

### DETAILED DESCRIPTION

The present invention generally relates to injectable electronics. In some aspects, the present invention is generally directed to systems and methods for interfacing an electrical cable with electrical elements, such as nanoscale wires, that are injected or otherwise introduced into a subject. The subject may be living or non-living. In one set of embodiments, electrical elements introduced within a subject may be placed in electrical communication to a circuit board using a plurality of electrically isolated contacts that the circuit board can clamp or otherwise connect to. The electrical contacts may be in electrical communication with the electrical elements using a joining portion. The circuit board can also be connected to an electrical cable that can be attached, for example, to a computer. In some cases, the electrical cable can be attached or detached to or from the circuit board, e.g., without requiring additional surgeries or interventions into the subject, to allow electrical communication with the electrical elements. Certain embodiments of the invention are also generally related to systems and methods of making or using such devices, systems and methods of inserting such devices into a subject, kits including such device, or the like.

As mentioned, certain aspects of the present invention are generally directed to systems and methods for interfacing an electrical cable with one or more electrical elements, such as nanoscale wires, microscale wires, electrodes, or the like, that have been injected or otherwise introduced into a subject. The subject may be living or non-living. Examples of living subjects include, but are not limited to, humans or non-humans, for example, a mammal such as a cow, sheep, goat, horse, rabbit, pig, mouse, rat, dog, cat, a primate (e.g., a monkey, a chimpanzee, etc.), or the like. In some cases, the living subject is a non-mammal such as a bird, an amphibian, or a fish. In some embodiments, the living subject is genetically engineered. The electrical elements may be injected or introduced into certain organs within the living subject, for example, within the brain, spinal cord, heart, or another organ of the living subject. In some cases, the organ is one that is electrically active, although this is not required, for example, if electrical elements able to determine chemical properties (e.g., pH), mechanical properties, or the like are used. Other steps may also be used to facilitate access. For example, to access to the brain, a relatively small hole may be drilled into the skull to allow injection of the electrical elements to occur.

However, in some cases, the electrical elements may be inserted or otherwise introduced into a non-living subject. For example, the non-living subject may be an inanimate material, for example, comprising a polymer, a ceramic, a metal, or the like. The electrical elements may be introduced during formation of the subject, or inserted or otherwise introduced after formation of the subject. As a non-limiting example, the electrical elements may be introduced into subjects such as concrete, sand, or soil, e.g., for materials testing or monitoring of the subject during use (for example, to detect structure integrity, moisture, pH, or the like). As another example, the electrical elements may be introduced into a gel, such as a hydrogel. For example, cells may be cultured on the gel (or another suitable culture medium), and changes (e.g., mechanical strain, chemical degradation, etc.) may be monitored using electrical elements within the gel or other culture medium.

In some cases, after electrical elements of a device as discussed herein have been injected or otherwise introduced into a living or non-living subject, an external electrical cable (or other suitable connection) may be attached or detached to the device as desired, with minimal discomfort to the subject (if the subject is living). A portion of the device may extend externally, out of the body of the subject, to facilitate connection. For example, a portion of the device may comprise a circuit board that the electrical elements are in electrical communication with, for example, through one or more joining portions. Accordingly, the electrical elements may be allowed to remain within the subject on an extended basis, with interactions with the device occurring externally of the subject in order to facilitate electrical communication with the electrical elements within the subject. For example, the electrical elements may be placed in electrical communication with a computer or other suitable device, for example, by attaching an electrical cable to a portion of the device, such as a circuit board, that is external to the subject. The introduction of the electrical elements into a subject may be performed via injection, implantation, insertion, or other techniques such as those described herein. In some cases, the introduction may be performed surgically, e.g., if the subject is living. In some embodiments, the electrical elements are injected into a subject, e.g., via a needle or a syringe. Examples of suitable techniques for introducing electrical elements into a subject include those described below, as well as those described in Int. Pat. Apl. Pub. Nos. WO 2015/084805, WO 2015/199784, and WO 2017/024154, each of which is incorporated herein by reference in its entirety. In some cases, as mentioned, the electrical elements of the device may be introduced into the subject, while a portion of the device is positioned outside of the subject, e.g., such that the device is accessible externally of the subject. In some embodiments, a portion of the device may first be introduced into the subject (for example, using a syringe), then connected to another portion of the device, e.g., one that is to be accessible externally of the subject.

Thus, the electrical elements positioned within the subject may be electrically connected to a device that can be attached to and detached from an external electrical cable as needed. For instance, a cable may be attached to the device when sensing and/or stimulation of the subject is desired, while the cable may be detached afterwards. Multiple or repeated attachments and detachments may occur, while the injected electrical elements remain in the subject. In some embodiments, due to the presence of the device, sensing and/or stimulating a subject may be performed as readily as attaching or detaching a cable to a suitable interface on the device. In contrast, many prior art techniques may not be sufficiently robust to permit multiple or repeated attachments and detachments, e.g., due to the delicacy of the electrical elements and/or the lack of a suitably robust interface available for connecting a suitable external electrical cable.

The electrical elements may form a first portion of a device, optionally with other elements (for example, connecting wires, polymers, metals, or the like). The device may also include a second portion containing one or more contacts. Optionally, the device may also include a joining portion that joins the first portion and the second portion. In some cases, more than one electric circuit may be present within the device, e.g., different contacts may be in electrical communication with different electrical elements, and in some cases, the electrical elements are individually addressable via the various contacts. In some cases, the second portion may be connected to a circuit board to form the device, e.g., using clamps. Electrical cables can then be attached and detached from the device as needed.

The electrical elements may be positioned or defined within the first portion of the device in any suitable arrangement. In some cases, the first portion of the device may be injected or otherwise introduced into a living or non-living subject as a single unit, although in other embodiments, different electrical elements may first be injected or otherwise introduced, then joined together to form the first portion of the device. Thus, the electrical elements, in some cases, may not be physically connected to each other. The electrical elements may define one, or more than one electrical circuit, in various embodiments. For instance, in some embodiments, some or all of the electrical elements are individually addressable. In addition, in certain embodiments, the electrical elements may form portions of transistor, e.g., as discussed below. For instance, an electrical element may act as a gate in a field effect junction.

The electrical elements may also be injected or otherwise introduced into a subject, as mentioned. If more than one electrical element is introduced, the electrical elements may each independently be the same or different. Examples of electrical elements include, but are not limited to, the following. In one set of embodiments, for instance, some or all of the electrical elements may be nanoscale electrical elements and/or or microscale electrical elements, such as those described in detail below. For example, nanoscale electrical elements may comprise nanowires and/or nanotubes. Other electrical elements, including those larger than the nanoscale, may also be used in certain cases, for example, microscale wires (e.g., having one or more cross-sectional dimensions of less than 1 mm, but being larger than a nanoscale wire).

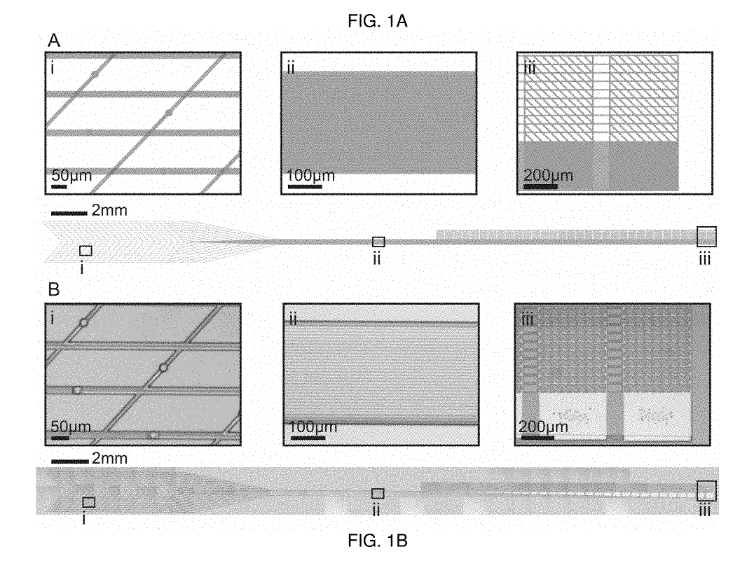

A variety of arrangements and configurations of electrical elements are possible within the first portion, e.g., to define one or more electrical circuits. For example, the electrical elements may each independently be connected in series, in parallel, or in a mesh, and/or isolated from each other. As an illustrative non-limiting example, a plurality of electrical elements may be arranged within a grid or mesh, e.g., of filaments. Thus, the mesh may be formed from one or more filaments, and some or all of the filaments within the mesh may include one or more electrical elements. In some cases, the grid or mesh may be substantially regularly arranged, for example, in a rectangular or parallelogram pattern, e.g., as shown in FIG. 1A. (It should be understood that, once inserted into a subject, the mesh may adopt other, distorted configurations, although topologically, the filaments stay within their relative positions within the mesh.) In addition, some or all of the filaments within the mesh may include one or more electrical elements, such as nanotubes or nanowires, including those discussed herein. However, the filaments within the mesh need all not necessarily include such electrical elements. The filaments within the mesh may independently each contain conductive portions (for example, metals), and/or semiconducting portions (for example, silicon nanowires), and/or insulating portions (for example, polymers), such as those discussed in more detail herein. The filaments within the mesh may include nanoscale filaments and/or microscale filaments, although in other cases, the filaments within the mesh may include filaments larger than the nanoscale or microscale, e.g., in addition to or instead of nanoscale filaments and/or microscale filaments.

The mesh, if present, may have any regular periodic arrangement of filaments. In some cases, the filaments are substantially straight. In some embodiments, if two or more substantially parallel groups of filaments form a mesh, the parallel groups may be arranged in any suitable angles relative to each other. In addition, filaments in one group may have the same or different spacings or periodicities as filaments in other groups. The filaments also may independently have the same or different average diameters relative to each other.

Thus, for example, a group of filaments may be spaced or have repeat units such that the filaments of that group have an average spacing or periodicity of at least 1 micrometer, at least 2 micrometers, at least 3 micrometers, at least 5 micrometers, at least 10 micrometers, at least 20 micrometers, at least 30 micrometers, at least 50 micrometers, at least 60 micrometers, at least 100 micrometers, at least 200 micrometers, at least 300 micrometers, at least 500 micrometers, at least 1 mm, etc. The filaments may also be spaced or have repeat units such that the filaments have an average spacing of no more than 1 mm, no more than 500 micrometers, no more than 300 micrometers, no more than 200 micrometers, no more than 100 micrometers, no more than 600 micrometers, no more than 50 micrometers, no more than 30 micrometers, no more than 20 micrometers, no more than 10 micrometers, no more than 5 micrometers, no more than 3 micrometers, no more than 2 micrometers, or no more than 1 micrometer. Combinations of any of these spacings or periodicities are also possible in various embodiments; for example, the filaments within a group may have an average spacing of between 10 micrometers and 30 micrometers, or between 200 micrometers and 500 micrometers, etc.

If two groups of filaments meet, they may meet at any suitable angle. In one embodiment, the two groups of filaments are orthogonal to each other, e.g., meeting at an angle of about 90°. However, other angles are also possible. For example, the angle may be 5° or more, 10° or more, 15° or more, 20° or more, 25° or more, 30° or more, 35° or more, 40° or more, 45° or more, 50° or more, 55° or more, 60° or more, 65° or more, 70° or more, 75° or more, 80° or more, or 85° or more. The angle may also be 90° or less, 85° or less, 80° or less, 75° or less, 70° or less, 65° or less, 60° or less, 55° or less, 50° or less, 45° or less, 40° or less, 35° or less, 30° or less, 25° or less, 20° or less, 15° or less, 10° or less, or 5° or less. Combinations of any of these angles are also possible in some embodiments, e.g., the filaments may meet an angle of between 30° and 45°, or between 60° and 65°. In addition, it should be understood that in some embodiments, a mesh may have various groups of filaments meeting at various angles, e.g., the mesh need not have only a single angle.

In some embodiments, the device may have a joining portion connecting a first portion of the device (e.g., comprises one or more electrical elements such as nanoscale wires and/or microscale wires) to a second portion of the device (e.g., comprising one or more electrical contacts).

In one set of embodiments, the joining portion may have one or more electrical connections passing through and connecting the first portion to the second portion of the device. See, e.g., FIG. 1A. The electrical connections may comprise, for example, metals or semiconductors, such as those described herein. For instance, metals may include aluminum, gold, silver, copper, molybdenum, tantalum, titanium, nickel, tungsten, chromium, palladium, platinum, as well as any combinations of these and/or other metals, and semiconductors may include silicon, gallium, germanium, diamond (carbon), tin, selenium, tellurium, boron, phosphorous, and/or other semiconductors (including elemental and compound semiconductors). In some cases, the electrical connections are parallel to each other. The electrical connections may be isolated from each other in some cases, for example, separated by an insulating material (for example, a photoresist such as SU-8, or polymers including those described herein). In some cases, the joining portion comprises a biocompatible material.

The joining portion can have any suitable length. In some cases, the length may depend, at least in part, on the expected depth of injection or other introduction of electrical elements within a living or non-living subject, e.g., such that at least a portion of the second portion of the device is able to remain externally of the subject after introduction of the electrical elements. For instance, the length of the joining portion may be less than 100 cm, less than 80 cm, less than 75 cm, less than 70 cm, less than 65 cm, less than 60 cm, less than 55 cm, less than 50 cm, less than 45 cm, less than 40 cm, less than 30 cm, less than 25 cm, less than 20 cm, less than 10 cm, less than 5 cm, less than 3 cm, or less than 1 cm. In some cases, the length may be at least 1 cm, at least 3 cm, at least 5 cm, at least 10 cm, at least 15 cm, at least 20 cm, at least 25 cm, at least 30 cm, at least 35 cm, at least 40 cm, at least 45 cm, at least 50 cm, at least 55 cm, at least 60 cm, at least 65 cm, at least 70 cm, at least 75 cm, at least 80 cm, at least 100 cm, etc. Combinations of any of these are also possible; for example, the length may be between 10 cm and 20 cm. In some cases, the joining portion may have a maximum cross-sectional dimension that is less than 5 cm, less than 4 cm, less than 3 cm, less than 2 cm, less than 1 cm, less than 5 mm, less than 3 mm, less than 2 mm, less than 1 mm, less than 500 micrometers, less than 300 micrometers, less than 200 micrometers, less than 100 micrometers, less than 50 micrometers, less than 30 micrometers, less than 20 micrometers, less than 10 micrometers, less than 5 micrometers, less than 3 micrometers, less than 2 micrometers, less than 1 micrometer, etc.

The joining portion may be made from materials similar to those in the first portion of the device containing electrical elements (for example, comprising the same metals, the same polymers, etc.) and/or fabricated using the same or different techniques as those for forming the first portion. In some cases, both the first portion and the joining portion are fabricated simultaneously.

As mentioned, the device may also have a second portion that comprises one or more contacts. These contacts may be used to connect the device to, for example, a circuit board. In some cases, more than one electrical connection to the circuit board may be desired, and thus, in some cases, there may be a plurality of electrically isolated contacts on the second portion, e.g., a connection to one contact may be independent of another contact (although it should be understood that one or more electrically isolated contacts may be part of the same electrical circuit in some cases, e.g., one may act as a positive and the other as a negative or a ground, etc.). The electrical contacts may thus facilitate electrical communication between the electrical elements and the circuit board.

Any number of electrical contacts may be used within the second portion of the device. For example, the device may have at least 2, at least 3, at least 4, at least 5, at least 6, at least 7, at least 8, at least 12, at least 16, at least 20, at least 24, at least 32, at least 36, at least 40, at least 45, at least 50, at least 64, at least 100, at least 128, at least or more contacts.

In some embodiments, the contacts may be regularly spaced, e.g., within the device. This may be useful, for example, to allow for connection to the circuit board. For instance, a plurality of such contacts may be useful to allow at least some of the electrical elements to be individually addressable. For instance, the contacts may have a spacing or gap between the contacts of at least 0.2 mm, at least 0.3 mm, at least 0.4 mm, at least 0.5 mm, at least 0.6 mm, at least 0.7 mm, at least 0.8 mm, at least 0.9 mm, or at least 1 mm and/or no more than 1.1 mm, no more than 1.0 mm, no more than 0.9 mm, no more than 0.8 mm, no more than 0.7 mm, no more than 0.6 mm, no more than 0.5 mm, no more than 0.4 mm, no more than 0.3 mm, or no more than 0.2 mm between the contacts. In some cases, combinations of any of these are also possible; for example, the spacing may be between 0.4 mm and 0.6 mm, between 0.9 mm and 1.1 mm, between 0.2 mm and 0.4 mm, etc. In some cases, the spacing may be about 0.5 mm or about 1 mm.

The contacts may have any suitable shape and/or size. For instance, the contacts may be square or rectangular in certain cases. The contacts may independently have substantially the same size, or different sizes. In some cases, the contacts may have an average area of at least 0.1 mm², at least 0.2 mm², at least 0.3 mm², at least 0.4 mm², at least 0.5 mm², at least 0.6 mm², at least 0.7 mm², at least 0.8 mm², at least 0.9 mm², at least 1 mm², at least 1.1 mm², at least 1.2 mm², at least 1.3 mm², at least 1.4 mm², at least 1.5 mm², at least 2 mm², at least 3 mm², at least 4 mm², at least 5 mm², etc. In some cases, the contacts may have an average area of no more than 10 mm², no more than 5 mm², no more than 4 mm², no more than 3 mm², no more than 2 mm², no more than 1.5 mm², no more than 1.4 mm², no more than 1.3 mm², no more than 1.2 mm², no more than 1.1 mm², no more than 1.0 mm², no more than 0.9 mm², no more than 0.8 mm², no more than 0.7 mm², no more than 0.6 mm², no more than 0.5 mm², no more than 0.4 mm², no more than 0.3 mm², no more than 0.2 mm², no more than 0.1 mm², etc. Combinations of any of these are also possible. For instance, the contacts may have an average area per contact of between 0.2 mm² and 1.2 mm².

In addition, the contacts may have any suitable width, and the contacts may independently have substantially the same width, or different widths. For instance, the width may be at least 0.2 mm, at least 0.3 mm, at least 0.4 mm, at least 0.5 mm, at least 0.6 mm, at least 0.7 mm, at least 0.8 mm, at least 0.9 mm, or at least 1 mm and/or no more than 1.1 mm, no more than 1.0 mm, no more than 0.9 mm, no more than 0.8 mm, no more than 0.7 mm, no more than 0.6 mm, no more than 0.5 mm, no more than 0.4 mm, no more than 0.3 mm, or no more than 0.2 mm. In some cases, combinations of any of these are also possible; for example, the width of the contacts may be between 0.4 mm and 0.6 mm, between 0.9 mm and 1.1 mm, between 0.2 mm and 0.4 mm, etc.

In some cases, the width of the contacts and/or the spacing between the contacts within the device may be chosen such that, when connecting it to a circuit board (e.g., using a suitable connector) or an electrical cable, the possibility that two contacts will be in electrical communication with each other due to the electrical contacts within the connector or cable is minimized or eliminated, i.e., to reduce or eliminate the possibility of creating a short circuit when attaching the connector or cable to the contacts.

As a non-limiting illustrative example, if a cable has a series of electrical contacts with a width of 0.3 mm and a spacing between contacts of 0.2 mm (i.e., a periodicity of 0.5 mm), then the contacts in the device may be designed to have a width of 0.2 mm and a spacing between contacts of 0.3 mm (for a periodicity of 0.5 mm), such that it is difficult or impossible to position a contact of the device precisely in the gap between the two adjacent electrical contacts of the cable that would create a short between the contacts.

In some cases, the contacts are made from materials similar to those in the first portion of the device containing electrical elements (for example, comprising the same metals, the same polymers, etc.) and/or fabricated using the same or different techniques as those for forming the first portion. For example, the contacts may comprise metals such as aluminum, gold, silver, copper, molybdenum, tantalum, titanium, nickel, tungsten, chromium, palladium, platinum, as well as any combinations of these and/or other metals. In some cases, the second portion is fabricated simultaneously as the first portion and/or the joining portion. However, in other cases, the contacts are not made from materials similar to those in the first portion or joining portion of the device. In addition, in some (but not all) embodiments, the contacts may comprise electrical elements such as nanoscale wires and/or microscale wires, or other elements such as those described herein. Non-limiting examples of suitable materials include any of those described below.

Thus, in one set of embodiments, the contacts may be formed from meshes similar to those discussed above (including having the dimensions and/or materials previously discussed above). Accordingly, in some cases, the filaments within the mesh may include nanoscale filaments and/or microscale filaments, although in other cases, the filaments within the mesh may include filaments larger than the nanoscale or microscale, e.g., in addition to or instead of nanoscale filament sand/or microscale filaments.

For example, a plurality of filaments may be formed into groups of filaments having a regular periodic arrangement of filaments. For example, the filaments may be substantially straight. In some embodiments, if two or more substantially parallel groups of filaments form a mesh, the parallel groups may be arranged in any suitable angles relative to each other. In addition, filaments in one group may have the same or different spacings or periodicities as filaments in other groups. The filaments also may independently have the same or different average diameters relative to each other. It should be understood that if meshes are used as the contacts, they may have the same or different dimensions and/or materials as meshes of the first portion (if meshes are present).

Thus, for example, a group of filaments may be spaced or have repeat units such that the filaments of that group have an average spacing or periodicity of at least 1 micrometer, at least 2 micrometers, at least 3 micrometers, at least 5 micrometers, at least 10 micrometers, at least 20 micrometers, at least 30 micrometers, at least 50 micrometers, at least 100 micrometers, at least 200 micrometers, at least 300 micrometers, at least 500 micrometers, at least 1 mm, etc. The filaments may also be spaced or have repeat units such that the filaments have an average spacing of no more than 1 mm, no more than 500 micrometers, no more than 300 micrometers, no more than 200 micrometers, no more than 100 micrometers, no more than 50 micrometers, no more than 30 micrometers, no more than 20 micrometers, no more than 10 micrometers, no more than 5 micrometers, no more than 3 micrometers, no more than 2 micrometers, or no more than 1 micrometer. Combinations of any of these spacings or periodicities are also possible in various embodiments; for example, the filaments within a group may have an average spacing of between 10 micrometers and 30 micrometers, or between 200 micrometers and 500 micrometers, etc.

If two groups of filaments meet, they may meet at any suitable angle. In one embodiment, the two groups of filaments are orthogonal to each other, e.g., meeting at an angle of about 90°. However, other angles are also possible. For example, the angle may be 5° or more, 10° or more, 15° or more, 20° or more, 25° or more, 30° or more, 35° or more, 40° or more, 45° or more, 50° or more, 55° or more, 60° or more, 65° or more, 70° or more, 75° or more, 80° or more, or 85° or more. The angle may also be 90° or less, 85° or less, 80° or less, 75° or less, 70° or less, 65° or less, 60° or less, 55° or less, 50° or less, 45° or less, 40° or less, 35° or less, 30° or less, 25° or less, 20° or less, 15° or less, 10° or less, or 5° or less. Combinations of any of these angles are also possible in some embodiments, e.g., the filaments may meet an angle of between 30° and 45°, or between 60° and 65°. In addition, it should be understood that in some embodiments, a mesh may have various groups of filaments meeting at various angles, e.g., the mesh need not have only a single angle.

However, it should be understood that meshes are not required in all embodiments for the contacts of the second portion. The contacts may have any shape or structure, for example, square, rectangular, circular, trapezoidal, or the like. In some instances, the contacts have a shape or structure that allows a suitable electrical connection to be made to the contact, e.g., such that the contact is in electrical connection with a circuit board, an electrical cable, or the like. For example, the contact may have a shape that allows a clamp connection or a crimp connection to be made between the contact and a circuit board. In some cases, for example, the contact may have a substantially solid structure, or a porous structure.

In one set of embodiments, the first portion and the second portion of the device may be introduced into a living or non-living subject using a syringe or a tube. In some cases, at least part of the second portion of the device may initially be collapsed to be able to fit through the syringe or a tube, then after introduction or injection of the first portion, the second portion may be expanded to allow connection, for example, to an electrical apparatus to form the device. For example, the contacts may be folded, curled, or otherwise mechanically manipulated so as to be able to fit.

In addition, in one set of embodiments, there may be a backing layer on some or all of the electrical contacts. The backing layer may be useful, e.g., to provide structural integrity to the contacts. In some cases, for example, the backing layer may comprise tape, such as dicing tape or electrical tape, or the backing layer may comprise a polymer, such as polyvinyl chloride, polyethylene, a polyolefin, or the like. In some embodiments, the backing layer to the contacts may be applied after introduction of the electrical elements to the subject, for example, after the contacts have passed through a tube or syringe. However, it should be understood that a backing layer is not required on all of the electrical contacts.

In certain embodiments, the electrical contacts may have conductive materials on both sides, which may facilitate connection to a circuit board. This may be useful, for example, since a physical connection thus cannot be attached “upside down,” as either side of the electrical contact can be used. If a backing layer or other layer to provide structural integrity to the contacts, then the materials used to provide electrical contact may present on both sides of the electrical contacts. In addition, the same or different materials may be present on each side, and a variety of methods can be used to attach the conductive materials to those sides. Thus, for example, a contact may include a first side comprising a first metal (e.g., platinum), and a second metal that may be the same, or different than the first metal (e.g., gold). For instance, in one embodiment, the electrode may be a double-sided platinum electrode.

In some cases, the contacts may be physically connected to a circuit board (e.g., a printed circuit board), or other electrical apparatus, e.g., to produce an electrical connection between the contacts and the circuit board or other electrical apparatus. The electrical apparatus may have one or more suitable electrical connections for connection to the contacts of the second portion. For example, the electrical apparatus may have at least 2, at least 3, at least 4, at least 5, at least 6, at least 7, at least 8, at least 12, at least 16, at least 20, at least 24, at least 32, at least 36, at least 40, at least 45, at least 50, at least 64, at least 100, at least 128, at least or more electrical connections for connection to the contacts.

In some cases, the contacts may be made using one or more electrical connectors. Examples include solderless electrical connectors, for example, crimp connectors, clamp connectors, screws, or the like. As other examples, solder or other techniques could be used to electrically connect the contacts to the circuit board or other electrical apparatus, i.e., a connector is not necessarily required in all embodiments to connect the contacts to a circuit board or other electrical apparatus. In some embodiments, for example, contacts may be made directly to a cable, such as an FFC, which can be connected to a circuit board or other electrical apparatus. In certain cases, for instance, the electrical contacts may be made directly on the electrical contacts of a cable, wires, or the like, e.g., for connecting to a circuit board. For example, the contacts may be made by cold welding, or by forming the contacts directly on the pads of a cable, which may form a relatively low-resistance contact.

The type of connection may each independently be the same or different for each contact. In some cases, the circuit board (or other electrical apparatus) may have a spacing of electrical connections that substantially matches the contacts, e.g., to allow connection between the electrical connections and the contacts in one-to-one correspondence.

Circuit boards and other similar electrical apparatuses may be custom-made, or a variety of circuit boards can be readily obtained commercially, e.g., having various methods for connecting to electrical contacts, including clamp or crimp connectors. Circuit boards and other similar electrical apparatuses may also be obtained in a variety of sizes and materials. In some cases, the circuit board (or other electrical apparatus) may have a maximum linear dimension of less than 50 cm, less than 40 cm, less than 30 cm, less than 25 cm, less than 20 cm, less than 15 cm, less than 10 cm, less than 5 cm, or less than 3 cm, and/or a weight of less than 1 kg, less than 500 g, less than 300 g, less than 100 g, less than 50 g, less than 30 g, less than 10 g, less than 5 g, less than 3 g, less than 2 g, or less than 1 g. For example, as shown in FIG. 1D, a printed circuit board may be used that has a size and weight such that it can be carried around by a mouse without substantial impairment (e.g., due to its size or weight). Thus, in some embodiments, the apparatus may have a size and/or weight that it can be carried around by a subject (e.g., if the subject is living, or mobile). The circuit board may also contain other functionalities, for example, digital multiplexing, wireless communications, signal processing, or the like.

In addition, in accordance with certain embodiments of the invention, the circuit board or other similar electrical apparatus may be immobilized relative to the subject, e.g., to facilitate portability and/or reduce impairment to the subject. For example, the apparatus may be directly immobilized onto a living subject, e.g., on the skin of the subject, or attached to a bone, the head, or other portion of the subject. For example, the apparatus may be attached to a living or non-living subject using cement (e.g., dental cement), cyanoacrylates, polymethylmethacrylates or other glues or adhesives, epoxy, and/or the apparatus may be screwed or otherwise immobilized onto the subject, e.g., using screws, wires, nails, or the like. The apparatus may also be immobilized on a more temporary basis, for example, using slings, wraps, fabric, string, magnets, or the like to immobilize the apparatus to the subject, for example, by tying or binding the apparatus to the subject.

In some embodiments, the apparatus may be protected in some fashion, for example, against the introduction of liquids, and/or attempts by the subject or others to remove the apparatus from the subject. For example, the apparatus may be protected by adding epoxy, cement, or other materials (such as those described above) to prevent or discourage removal of the apparatus from the subject (e.g., due to scratching, biting, rubbing against a wall, etc.), and/or to prevent or limit the entry of water or other liquids, for example, by covering some or all of the apparatus.

It can be relatively difficult to introduce electrical elements into a subject and also attach them to a computer or other external electrical device, e.g., due to their size (e.g., for nanoscale electrical elements), fragility, and/or the difficulty in introducing them to a subject, for instance, if the subject is alive or mobile. However, it may be relatively easier to attach an electrical cable to a circuit board. Accordingly, in some embodiments, various electrical elements can be introduced into a subject and attached to a circuit board or other suitable apparatus (which may, in some cases, be immobilized to the subject) to form a device to which can be placed in electrical communication with a computer or other external electrical device as needed, for example, by attaching and detaching an electrical cable (or other suitable connection) to the device, e.g., by the circuit board or other suitable apparatus.

In some embodiments, the electrical cable (or other suitable connection) may be repeatedly attached and detached to the device, e.g., to a circuit board. For example, when an electrical connection (e.g., with a computer or other external electrical device) is needed (for example, to determine a physical or electrical property of the subject, or to apply an electrical stimulus to the subject, etc.), an electrical cable may be attached as desired, e.g., without requiring subsequent surgeries or actions need to inject additional electrical elements or wires into the subject. In some embodiments, e.g., using relatively standard electrical connectors that are widely used commercially, electrical cables may be quickly attached or detached to the device as needed. The same or different electrical cables (and/or computers or other external electrical devices) may be used. In some cases, the electrical cable may be left attached to the device for relatively long periods of time, e.g., at least an hour, at least a day, at least 2 days, at least 3 days, at least a week, etc. In other cases, however, the attachment may be relatively short, e.g., no more than a day or no more than an hour.

A variety of standard electrical cables may be used in various embodiments, including ribbon cables or flexible flat cables having any number of channels or wires, e.g., 4, 6, 8, 9, 10, 14, 15, 16, 18, 20, 24, 25, 26, 34, 37, 40, 50, 60, 64, 80, etc. The cable may also have any pitch or spacing, e.g., a spacing of 0.25 mm, 0.3 mm, 0.5 mm, 0.625 mm, 0.635 mm, 0.8 mm, 1 mm, 1.25 mm, 1.27 mm, 2 mm, 2.54 mm, or the like. Other types of cables may also be used in various embodiments, e.g., individual cables, twisted-pair wires, or the like. The circuit board may have any suitable connector for connecting the cables, and in some cases, the connector may be one that can be used for repeated connection and disconnection. Many such connector types are commercially available, such as microribbon connectors, BT224 connectors, Omnetics connectors, or the like.

The electrical cable may, in turn, be attached and detached to a suitable external electrical device, such as a computer, an oscilloscope, a voltage amplifier, a current amplifier, electronic test equipment, a voltmeter, an ohmmeter, an ammeter, a multimeter, a signal generator, a pulse generator, a power supply, a test probe, a monitor, or the like.

Such devices may be used, for example, to determine one or more electrical elements within the first portion of the device, and/or to apply a signal (e.g., an electrical signal) to one or more electrical elements within the first portion of the device. Determinations may be qualitative and/or quantitative, depending on the application. In addition, in some cases, as previously discussed, one or more of the electrical elements within the first portion may be independently electrically addressable. In some cases, for example, different electrical elements may be placed in electrical communication with different external electrical devices, and/or more than one external electrical device may be placed in electrical communication with an electrical element within a living or non-living subject.

In addition, it should be understood that in other embodiments, other connections are possible besides electrical connections through electrical cables. For example, other types of connections are also possible, e.g., such that an external electrical device, such as a computer, is in electrical communication with one or more of the electrical elements within the subject. For example, the circuit board or other apparatus may be able to create a connection using wireless technologies, such as using light, infrared radiation, radio waves, magnetic pulses, or the like. A variety of wireless components that may be present within a circuit board to facilitate such communications are readily available commercially, and include standards such as LTE, LTE-Advanced, Wi-Fi, Bluetooth, or the like. As another example, light may be used instead of an electrical connection, for example, by using fiber optic cables to connect the circuit board (or other electrical apparatus) to a computer or other external electrical device. For example, in one embodiment, the first portion of the device (e.g., within the subject) may contain optoelectronic devices. In some cases, the joining portion may also contain waveguides, e.g., that can be connected to a circuit board or other electrical apparatus as discussed herein.

As mentioned, in one set of embodiments, electrical elements may be introduced into a living or non-living subject via injection, for example through a tube or a syringe. Thus, in certain aspects, at least a portion of a device as discussed herein may be positioned in a tube, such as a metal tube. For example, a first portion (e.g., comprises one or more electrical elements such as nanoscale wires and/or microscale wires), a second portion (e.g., comprising one or more electrical contacts), and optionally a joining portion may be contained within a tube for injection into a subject. After injection or introduction into a subject, a circuit board or other electrical apparatus may be attached to the second portion, e.g., using one or more electrical contacts, as discussed above.

In some cases, the portions of the device within the tube may be shaped to be cylindrical or curved, and/or the portions may be compressed to fit inside the tube, although the device may be able to expand after exiting the tube or additional component attached, e.g., as discussed herein. The tube may be formed out of any suitable material. For instance, the tube may comprise stainless steel. The tube may also be other materials in other embodiments. For example, the tube may be plastic, or the tube may be glass. The tube may be a needle or form part of a syringe, or the tube may be form part of an injector device, such as a microinjector. In some cases, the tube is cylindrical, although the tube may be noncylindrical in other cases. For instance, the tube may be tapered or beveled in some embodiments. In some cases, the tube is hollow. In some cases, the tube has a circular cross-section. However, in other cases, the tube may not have a circular cross-section. For example, the tube may have a square or rectangular cross-section, or the tube may have an open cross-section, e.g., having a “U”-shaped cross section. The tube may have any suitable inner diameter. For instance, the tube may have an inner diameter of less than about 1.2 mm, less than about 1 mm, less than about 800 micrometers, less than about 600 micrometers, less than about 500 micrometers, less than about 400 micrometers, less than about 300 micrometers, less than about 200 micrometers, less than about 100 micrometers, less than about 80 micrometers, less than about 60 micrometers, less than about 50 micrometers, etc.

The portions of the device may pass through the tube using any suitable method. The portions may fully pass through the tube, or in some cases, the portions may only partially pass through the tube such that part of the device remains within the tube. The portions may be fully or partially expelled or urged from the tube using suitable forces, pressures, mechanisms, or apparatuses. For instance, in one set of embodiments, the portions may be expelled using a microinjection device. In another embodiment, the portions may be manually expelled, e.g., by pushing the plunger of a syringe. In some cases, fluids (liquids or gases) may be used to expel the device. For instance, water, saline, or air may be added to the tube to assist in expulsion. In some cases, a pump or other fluid source (e.g., a spigot or a tank) may be used to introduce fluid into the tube. In some cases, a relatively small amount of fluid may be used. For instance, the amount of fluid used to expel the device may be less than 1 ml, less than 500 microliters, less than 300 microliters, less than 200 microliters, less than 100 microliters, less than 50 microliters, less than 30 microliters, less than 20 microliters, or less than 10 microliters. For instance, a pump may pump fluid into the tube (or through tubing or other fluidic channels) into the tube to cause portions of the device to be expelled therefrom (e.g., partially or fully). The portions injected into the subject may be injected at a controlled rate and/or with controllable position, for example, by controlling the pressure or flow rate of fluid from the pump. In some cases, the tube may be inserted into a target such that portions of the device are expelled directly into the target. For example, the tube may be inserted into a subject, e.g., into the tissue of a subject, such as those described herein. In another embodiment, the tube may be inserted into soft matter. For instance, the tube may be inserted into a polymer or a gel. Thus, the device may be expelled from the tube such that the device at least partially penetrates into the target, e.g., the first portion of the device containing electrical elements.

As mentioned, in some cases, the portions of the device, when inserted into the tube, is constrained or compressed in some fashion such that, upon expulsion (fully or partially), those portions are able to at least partially expand. As a non-limiting example, the device may include a network that is rolled to form a cylinder (for example, a mesh containing electrical elements); upon expulsion, those portions are able to at least partially unroll and expand. In some cases, the portions are able to spontaneously expand, e.g., upon exiting the tube. The expansion may occur rapidly, or on longer time scales. As another example, the portions may unfold, or portions may uncompress, upon exiting a tube. The portions may expand to reach its original shape. In some cases, the portions may substantially return to their original shape after about 24 hours, after about 48 hours, or after about 72 hours. In certain embodiments, it may take longer for the portions to substantially return to its original shape, e.g., after 1 week, after 2 weeks, after 3 weeks, after 4 weeks, after 5 weeks, after 6 weeks, etc. In some cases, however, the portions may not necessarily return to its original shape, e.g., inherently, and/or due to the matter that the portions was injected or introduced into. For example, the presence of tissue (or other matter) may prevent the portions from fully expanding back to its original shape after insertion.

In addition, in some embodiments of the invention, the portions may be expelled or urged from a tube (or other suitable carrier) such that the portions are not significantly distorted, e.g., due to mechanical resistance offered by the medium that the portions are being inserted into. In some embodiments, the portions may be expelled or urged from a tube without substantially altering the position of those portions, relative to the medium. This may be useful, for example, to prevent or minimize compressive forces on those portions as it encounters the medium, e.g., which may deform or “crumple” those portions.

In some cases, those portions may be “at rest” relative to the medium while the tube is removed. In other embodiments, however, there may be some relative motion, e.g., due to forces involved in removing the tube and/or urging the portions out of the tube, movement of the medium (e.g., if the medium is alive), etc. In some cases, the motion may be less than about 10 cm/s, less than about 5 cm/s, less than about 3 cm/s, less than amount 1 cm/s, less than about 5 mm/s, less than about 3 mm/s, less than about 1 mm/s, less than about 0.5 mm/s, less than about 0.3 mm/s, or less than about 0.1 mm/s. Thus, the position of those portions, relative to the medium, may not change substantially, or the position may change by no more than about 40%, no more than about 35%, no more than about 30%, no more than about 25%, no more than about 20%, no more than about 15%, no more than about 10%, no more than about 5%, no more than about 2%, or no more than about 1%, relative to the length of those portions. In another set of embodiments, the position of those portions of the device, relative to the medium, may change by no more than about 1 mm, no more than about 800 micrometers, no more than about 500 micrometers, no more than about 400 micrometers, no more than about 300 micrometers, no more than about 200 micrometers, no more than about 100 micrometers, no more than about 80 micrometers, no more than about 50 micrometers, no more than about 30 micrometers, no more than about 20 micrometers, no more than about 10 micrometers, no more than about 5 micrometers, etc.

This may be accomplished, for example, by withdrawing the tube from the medium while simultaneously urging the portions of the device out of the tube, e.g., such that these rates are substantially comparable. In some cases, the rates may differ by no more than about 40%, no more than about 35%, no more than about 30%, no more than about 25%, no more than about 20%, no more than about 15%, no more than about 10%, no more than about 5%, no more than about 2%, or no more than about 1%, relative to the slower of the two rates. In one embodiment, the rates are substantially equal.

As another example, the tube may be removed from the medium by dissolving or liquefying the tube. For example, the tube may be formed from frozen saline, or another suitably benign (or biocompatible) material, and after insertion, the tube is simply allowed to melt while within the medium, thereby leaving those portions of the device behind without substantially altering its position, relative to the medium. As another example, the tube may be formed from a biodegradable polymer, such as polylactic acid, polyglycolic acid, polycaprolactone, etc.

In some aspects, other materials may also be present within the tube, e.g., in addition to the portions of the device. For example, in one set of embodiments, a gas or a liquid may be present within the tube. For instance, the tube may contain a liquid to facilitate expulsion of the device, or a liquid to assist in movement of the portions of the device out of the tube, or into the target. For instance, the tube may include a liquid such as saline, which can be injected into a living or non-living subject, e.g., along with the device. In addition, in some cases, the fluid may also contain one or more cells, which may be inserted or injected into a target along with those portions of the device. If the target is a living subject or biological tissue, the cells may be autologous, heterologous, or homologous to the tissue or to the subject.

In certain aspects, as mentioned, the device may comprise one or more electrical networks comprising electrical elements and/or conductive pathways in electrical communication with the electrical elements. For example, a first portion of a device may comprise one or more electrical elements, and one or more electrical networks in electrical communication with those electrical elements. These may be in electrical communication with one or more contacts in a second portion of the device, e.g., for attachment to a circuit board or other electrical apparatus, optionally via a joining portion. Accordingly, various portions of the device may include conductive pathways, separated by insulating materials, to allow such electrical communication to occur. In some cases, the insulating materials may also be biocompatible and/or biodegradable.

In some cases, at least some of the conductive pathways may also provide mechanical strength to portions of the device, and/or there may be polymeric or metal constructs that are used to provide mechanical strength to portions of the device. The same or different materials may be used in different portions of the device.

In some cases, a portion of the device (e.g., a first portion, a second portion, a mesh, etc.) may be flexible in some cases, e.g., the device may be able to bend or flex. For example, a portion may be bent or distorted by a volumetric displacement of at least about 5%, about 10%, or about 20% (relative to the undisturbed volume), without causing cracks and/or breakage within the device. For example, in some cases, the portion can be distorted such that about 5%, about 10%, or about 20% of the mass of the portion has been moved outside the original surface perimeter of the portion, without causing failure (e.g., by breaking or cracking of the portion, disconnection of portions of the electrical network, etc.). In some cases, portions of the device may be bent or flexed as described above by an ordinary human being without the use of tools, machines, mechanical device, excessive force, or the like. A flexible portion may be more biocompatible due to its flexibility, and the device may be treated as previously discussed to facilitate its insertion into a tissue.

In addition, a portion of the device may be non-planar in some cases, e.g., curved as previously discussed. For example, a portion of the device may be substantially U-shaped or cylindrical, and/or have a shape and/or size that is similar to a hypodermic needle. In some embodiments, a portion of the device (e.g., a first portion, a second portion, and/or a joining portion) may be generally cylindrical with a maximum outer diameter of no more than about 5 mm, no more than about 4 mm, no more than about 3 mm, no more than about 2 mm, no more than about 1 mm, no more than about 0.9 mm, no more than about 0.8 mm, no more than about 0.7 mm, no more than about 0.6 mm, no more than about 0.5 mm, no more than about 0.4 mm, no more than about 0.3 mm, or no more than about 0.2 mm. Accordingly, in some embodiments, the portions of the device may be able to be placed into a tube, e.g., of a needle or a syringe. As discussed herein, those portions of the device can then be inserted or injected out of the tube upon application of suitable forces and/or pressures, for instance, such that those portions can be inserted or injected into other matter. For instance, portions of the device may be injected into a living or non-living subject, e.g., a first portion containing one or more electrical elements.

In one aspect, the device may comprise a periodic structure comprising electrical elements. For example, the device may comprise a mesh or other two-dimensional array of electrical elements and/or other conductive pathways. The mesh may include a first set of conductive pathways, generally parallel to each other, and a second set of conductive pathways, generally parallel to each other. The first set and the second set may be orthogonal to each other, or they may cross at any suitable angle. For instance, the sets may cross at a 30° angle, a 45° angle, or a 60° angle, or any other suitable angle. Mesh structures of the device may be particularly useful in certain embodiments. For instance, in a mesh structure, due to the physical connections, it may be easier for the structure to maintain its topological configuration, e.g., of the electrical elements relative to each other. In addition, it may be more difficult for the structure to become adversely tangled. If a periodic structure is used, the period may be of any suitable length. For example, the length of a unit cell within the periodic structure may be less than about 500 micrometers, less than about 400 micrometers, less than about 300 micrometers, less than about 200 micrometers, less than about 100 micrometers, less than about 80 micrometers, less than about 60 micrometers, less than about 50 micrometers, etc.

In certain aspects, the device may contain one or more polymeric constructs, e.g., within a first portion, second portion, and/or joining portion. The polymeric constructs typically comprise one or more polymers, e.g., photoresists, biocompatible polymers, biodegradable polymers, etc., and optionally may contain other materials, for example, metal leads or other conductive pathway materials. The polymeric constructs may be separately formed then assembled into the device, and/or the polymeric constructs may be integrally formed as part of the device, for example, by forming or manipulating (e.g. folding, rolling, etc.) the polymeric constructs into a 3-dimensional structure that defines the device.

In one set of embodiments, some or all of the polymeric constructs have the form of fibers or ribbons. For example, the polymeric constructs may have one dimension that is substantially longer than the other dimensions of the polymeric construct. The fibers can in some cases be joined together to form a network or mesh of fibers. For example, a device may contain a plurality of fibers that are orthogonally arranged to form a regular network of polymeric constructs. However, the polymeric constructs need not be regularly arranged. The polymer constructs may have the form of fibers or other shapes. In general, any shape or dimension of polymeric construct may be used to form a device.

In one set of embodiments, some or all of the polymeric constructs have a smallest dimension or a largest cross-sectional dimension of less than about 5 micrometers, less than about 4 micrometers, less than about 3 micrometers, less than about 2 micrometers, less than about 1 micrometer, less than about 700 nm, less than about 600 nm, less than about 500 nm, less than about 300 nm, less than about 200 nm, less than about 100 nm, less than about 80 nm, less than about 50 nm, less than about 30 nm, less than about 10 nm, less than about 5 nm, less than about 2 nm, etc. A polymeric construct may also have any suitable cross-sectional shape, e.g., circular, square, rectangular, polygonal, elliptical, regular, irregular, etc. Examples of methods of forming polymeric constructs, e.g., by lithographic or other techniques, are discussed below. In one set of embodiment, the polymeric constructs can be arranged such that the device is relatively porous, e.g., such that cells can penetrate into the device before and/or after insertion of the device. For example, in some cases, the polymeric constructs may be constructed and arranged within the device such that the device has an open porosity of at least about 30%, at least about 40%, at least about 50%, at least about 60%, at least about 70%, at least about 75%, at least about 80%, at least about 85%, at least about 90%, at least about 95%, at least about 97, at least about 99%, at least about 99.5%, or at least about 99.8%. The “open porosity” is generally described as the volume of empty space within the device divided by the overall volume defined by the device, and can be thought of as being equivalent to void volume. Typically, the open porosity includes the volume within the device to which cells can access. In some cases, the device does not contain significant amounts of internal volume to which the cells are incapable of addressing, e.g., due to lack of access and/or pore access being too small.

In some cases, a “two-dimensional open porosity” may also be defined, e.g., of a device that is subsequently formed or manipulated into a 3-dimensional structure. The two-dimensional open porosities of a device can be defined as the void area within the two-dimensional configuration of the device (e.g., where no material is present) divided by the overall area of device, and can be determined before or after the device has been formed into a 3-dimensional structure. Depending on the application, a device may have a two-dimensional open porosity of at least about 30%, at least about 40%, at least about 50%, at least about 60%, at least about 70%, at least about 75%, at least about 80%, at least about 85%, at least about 90%, at least about 95%, at least about 97, at least about 99%, at least about 99.5%, or at least about 99.8%, etc.

Another method of generally determining the two-dimensional porosity of the device is by determining the areal mass density, i.e., the mass of the device divided by the area of one face of the device (including holes or voids present therein). Thus, for example, in another set of embodiments, the device may have an areal mass density of less than about 100 micrograms/cm², less than about 80 micrograms/cm², less than about 60 micrograms/cm², less than about 50 micrograms/cm², less than about 40 micrograms/cm², less than about 30 micrograms/cm², or less than about 20 micrograms/cm².

The porosity of a device can be defined by one or more pores. Pores that are too small can hinder or restrict cell access. Thus, in one set of embodiments, the device may have an average pore size of at least about 100 micrometers, at least about 200 micrometers, at least about 300 micrometers, at least about 400 micrometers, at least about 500 micrometers, at least about 600 micrometers, at least about 700 micrometers, at least about 800 micrometers, at least about 900 micrometers, or at least about 1 mm. However, in other embodiments, pores that are too big may prevent cells from being able to satisfactorily use or even access the pore volume. Thus, in some cases, the device may have an average pore size of no more than about 1.5 mm, no more than about 1.4 mm, no more than about 1.3 mm, no more than about 1.2 mm, no more than about 1.1 mm, no more than about 1 mm, no more than about 900 micrometers, no more than about 800 micrometers, no more than about 700 micrometers, no more than about 600 micrometers, or no more than about 500 micrometers. Combinations of these are also possible, e.g., in one embodiment, the average pore size is at least about 100 micrometers and no more than about 1.5 mm. In addition, larger or smaller pores than these can also be used in a device in certain cases. Pore sizes may be determined using any suitable technique, e.g., through visual inspection (e.g., of microscope images), BET measurements, or the like.

In various embodiments, one or more of the polymers forming a polymeric construct may be a photoresist. While not commonly used in biological devices, photoresists are typically used in lithographic techniques, which can be used as discussed herein to form the polymeric construct. For example, the photoresist may be chosen for its ability to react to light to become substantially insoluble (or substantially soluble, in some cases) to a photoresist developer. For instance, photoresists that can be used within a polymeric construct include, but are not limited to, SU-8, 51805, LOR 3A, poly(methyl methacrylate), poly(methyl glutarimide), phenol formaldehyde resin (diazonaphthoquinone/novolac), diazonaphthoquinone (DNQ), Hoechst AZ 4620, Hoechst AZ 4562, Shipley 1400-17, Shipley 1400-27, Shipley 1400-37, or the like. These and many other photoresists are available commercially.

A polymeric construct may also contain one or more polymers that are biocompatible and/or biodegradable, in certain embodiments. A polymer can be biocompatible, biodegradable, or both biocompatible and biodegradable, and in some cases, the degree of biodegradation or biocompatibility depends on the physiological environment to which the polymer is exposed to.

Typically, a biocompatible material is one that does not illicit an immune response, or elicits a relatively low immune response, e.g., one that does not impair the device or the cells therein from continuing to function for its intended use. In some embodiments, the biocompatible material is able to perform its desired function without eliciting any undesirable local or systemic effects in a living subject. In some cases, the material can be incorporated into tissues within the subject, e.g., without eliciting any undesirable local or systemic effects, or such that any biological response by a living subject does not substantially affect the ability of the material from continuing to function for its intended use. For example, in a device, the device may be able to determine cellular or tissue activity after insertion, e.g., without substantially eliciting undesirable effects in those cells, or undesirable local or systemic responses, or without eliciting a response that causes the device to cease functioning for its intended use. Examples of techniques for determining biocompatibility include, but are not limited to, the ISO 10993 series for evaluating the biocompatibility of medical devices. As another example, a biocompatible material may be implanted in a living subject for an extended period of time, e.g., at least about a month, at least about 6 months, or at least about a year, and the integrity of the material, or the immune response to the material, may be determined. For example, a suitably biocompatible material may be one in which the immune response is minimal, e.g., one that does not substantially harm the health of the living subject. One example of a biocompatible material is poly(methyl methacrylate). In some embodiments, a biocompatible material may be used to cover or shield a non-biocompatible material (or a poorly biocompatible material) from the cells or tissue, for example, by covering the material.

A biodegradable material typically degrades over time when exposed to a biological system, e.g., through oxidation, hydrolysis, enzymatic attack, phagocytosis, or the like. For example, a biodegradable material can degrade over time when exposed to water (e.g., hydrolysis) or enzymes. In some cases, a biodegradable material is one that exhibits degradation (e.g., loss of mass and/or structure) when exposed to physiological conditions for at least about a month, at least about 6 months, or at least about a year. For example, the biodegradable material may exhibit a loss of mass of at least about 10%, at least about 20%, at least about 30%, at least about 40%, at least about 50%, at least about 60%, at least about 70%, at least about 80%, or at least about 90%. In certain cases, some or all of the degradation products may be resorbed or metabolized, e.g., into cells or tissues. For example, certain biodegradable materials, during degradation, release substances that can be metabolized by cells or tissues. For instance, polylactic acid releases lactic acid during degradation.

Examples of such biocompatible and/or biodegradable polymers include, but are not limited to, poly(lactic-co-glycolic acid), polylactic acid, polyglycolic acid, poly(methyl methacrylate), poly(trimethylene carbonate), collagen, fibrin, polysaccharidic materials such as chitosan or glycosaminoglycans, hyaluronic acid, polycaprolactone, and the like.

The polymers and other components forming the device can also be used in some embodiments to provide a certain degree of flexibility to the device, which can be quantified as a bending stiffness per unit width of polymer construct. In various embodiments, the overall device may have a bending stiffness of less than about 5 nN m, less than about 4.5 nN m, less than about 4 nN m, less than about 3.5 nN m, less than about 3 nN m, less than about 2.5 nN m, less than about 2 nN m, less than about 1.5 nN m, less than about 1 nN m, less than about 0.5 nM m, less than about 0.3 nM m, less than about 0.1 nM m, less than about 0.05 nM m, less than about 0.03 nM m, less than about 0.01 nM m, less than about 0.005 nM m, less than about 0.003 nM m, less than about 0.001 nM m, less than about 0.0005 nM m, less than about 0.0003 nM m, etc. In some cases, devices having relatively low bending stiffnesses are relatively flexible and bendable, and can be readily inserted into a tube, as discussed herein.

In some embodiments of the invention, the device may also contain other materials in addition to the photoresists or biocompatible and/or biodegradable polymers described above. Non-limiting examples include other polymers, growth hormones, extracellular matrix protein, specific metabolites or nutrients, or the like. For example, in one of embodiments, one or more agents able to promote cell growth can be added to the device, e.g., hormones such as growth hormones, extracellular matrix protein, pharmaceutical agents, vitamins, or the like. Many such growth hormones are commercially available, and may be readily selected by those of ordinary skill in the art based on the specific type of cell or tissue used or desired. Similarly, non-limiting examples of extracellular matrix proteins include gelatin, laminin, fibronectin, heparin sulfate, proteoglycans, entactin, hyaluronic acid, collagen, elastin, chondroitin sulfate, keratin sulfate, Matrigel™, or the like. Many such extracellular matrix proteins are available commercially, and also can be readily identified by those of ordinary skill in the art based on the specific type of cell or tissue used or desired.

As another example, in one set of embodiments, additional materials can be added to the device, e.g., to control the size of pores within the device, to promote cell adhesion or cell growth within the device, to increase the structural stability of the device, to control the flexibility of the device, etc. For instance, in one set of embodiments, additional fibers or other suitable polymers may be added to the device, e.g., electrospun fibers can be used as a secondary scaffold. The additional materials can be formed from any of the materials described herein, e.g., photoresists or biocompatible and/or biodegradable polymers, or other polymers described herein. As another non-limiting example, a glue such as a silicone elastomer glue or dental cement can be used to control the shape of the device.

In some cases, as mentioned the device can include a 2-dimensional structure that is formed into a final 3-dimensional structure, e.g., by folding or rolling the structure. It should be understood that although the 2-dimensional structure can be described as having an overall length, width, and height, the overall length and width of the structure may each be substantially greater than the overall height of the structure. The 2-dimensional structure may also be manipulated to have a different shape that is 3-dimensional, e.g., having an overall length, width, and height where the overall length and width of the structure are not each substantially greater than the overall height of the structure. For instance, the structure may be manipulated to increase the overall height of the material, relative to its overall length and/or width, for example, by folding or rolling the structure. Thus, for example, a relatively planar sheet of material (having a length and width much greater than its thickness) may be rolled up into a “tube,” such that the tube has an overall length, width, and height of relatively comparable dimensions.

Thus, for example, the 2-dimensional structure may comprise one or more electrical elements and one or more polymeric constructs formed into a 2-dimensional structure or network that is subsequently formed into a 3-dimensional structure. In some embodiments, the 2-dimensional structure may be rolled or curled up to form the 3-dimesional structure, or the 2-dimensional structure may be folded or creased one or more times to form the 3-dimesional structure. Such manipulations can be regular or irregular. In certain embodiments, as discussed herein, the manipulations are caused by pre-stressing the 2-dimensional structure such that it spontaneously forms the 3-dimensional structure, although in other embodiments, such manipulations can be performed separately, e.g., after formation of the 2-dimensional structure.

In some aspects, the device may include one or more metal leads or electrodes, or other conductive pathways. The metal leads or conductive pathways may provide mechanical support, and/or one or more metal leads can be used within a conductive pathway to an electrical element, such as a nanoscale wire and/or microscale wire. The metal lead may directly physically contact the electrical elements and/or there may be other materials between the metal lead and the electrical elements that allow electrical communication to occur. In some cases, one or more metal leads or other conductive pathways may extend such that the device can be connected to external electrical circuits, computers, or the like, e.g., using one or more electrical contacts as discussed herein. Metal leads are useful due to their high conductance, e.g., such that changes within electrical properties obtained from the conductive pathway can be related to changes in properties of the electrical elements, rather than changes in properties of the conductive pathway. However, it is not a requirement that only metal leads be used, and in other embodiments, other types of conductive pathways may also be used, in addition or instead of metal leads.

A wide variety of metal leads can be used, in various embodiments of the invention. As non-limiting examples, the metals used within a metal lead may include aluminum, gold, silver, copper, molybdenum, tantalum, titanium, nickel, tungsten, chromium, palladium, platinum, as well as any combinations of these and/or other metals. In some cases, the metal can be chosen to be one that is readily introduced into the device, e.g., using techniques compatible with lithographic techniques. For example, in one set of embodiments, lithographic techniques such as e-beam lithography, photolithography, X-ray lithography, extreme ultraviolet lithography, ion projection lithography, etc. may be used to layer or deposit one or more metals on a substrate. Additional processing steps can also be used to define or register the metal leads in some cases. Thus, for example, the thickness of a metal layer may be less than about 5 micrometers, less than about 4 micrometers, less than about 3 micrometers, less than about 2 micrometers, less than about 1 micrometer, less than about 700 nm, less than about 600 nm, less than about 500 nm, less than about 300 nm, less than about 200 nm, less than about 100 nm, less than about 80 nm, less than about 50 nm, less than about 30 nm, less than about 10 nm, less than about 5 nm, less than about 2 nm, etc. The thickness of the layer may also be at least about 10 nm, at least about 20 nm, at least about 40 nm, at least about 60 nm, at least about 80 nm, or at least about 100 nm. For example, the thickness of a layer may be between about 40 nm and about 100 nm, between about 50 nm and about 80 nm.

In some embodiments, more than one metal can be used within a metal lead. For example, two, three, or more metals may be used within a metal lead. The metals may be deposited in different regions or alloyed together, or in some cases, the metals may be layered on top of each other, e.g., layered on top of each other using various lithographic techniques. For example, a second metal may be deposited on a first metal, and in some cases, a third metal may be deposited on the second metal, etc. Additional layers of metal (e.g., fourth, fifth, sixth, etc.) may also be used in some embodiments. The metals can all be different, or in some cases, some of the metals (e.g., the first and third metals) may be the same. Each layer may independently be of any suitable thickness or dimension, e.g., of the dimensions described above, and the thicknesses of the various layers can independently be the same or different.

If dissimilar metals are layered on top of each other, they may be layered in some embodiments in a “stressed” configuration (although in other embodiments they may not necessarily be stressed). As a specific non-limiting example, chromium and palladium can be layered together to cause stresses in the metal leads to occur, thereby causing warping or bending of the metal leads. The amount and type of stress may also be controlled, e.g., by controlling the thicknesses of the layers. For example, relatively thinner layers can be used to increase the amount of warping that occurs.

Without wishing to be bound by any theory, it is believed that layering metals having a difference in stress (e.g., film stress) with respect to each other may, in some cases, cause stresses within the metal, which can cause bending or warping as the metals seek to relieve the stresses. In some embodiments, such mismatches are undesirable because they could cause warping of the metal leads and thus, the device. However, in other embodiments, such mismatches may be desired, e.g., so that the device can be intentionally deformed to form a 3-dimensional structure, as discussed below. In addition, in certain embodiments, the deposition of mismatched metals within a lead may occur at specific locations within the device, e.g., to cause specific warpings to occur, which can be used to cause the device to be deformed into a particular shape or configuration. For example, a “line” of such mismatches can be used to cause an intentional bending or folding along the line of the device.

The device may include one or more electrical elements, for example, in a first portion of the device, which may be the same or different from each other, in accordance with various aspects of the invention. In some cases, the electrical elements are nanoscale electrical elements, such as nanoscale wires, and/or microscale electrical elements, such as microscale wires. Non-limiting examples of such electrical elements are discussed in detail herein, and include, for instance, semiconductor wires (e.g., semiconductor nanowires or microwires), carbon nanotubes or microtubes, carbon fibers, organic electrical elements, or the like. In some cases, at least one of the electrical elements is a silicon nanowire. The electrical elements may also be straight, or kinked in some cases. In some embodiments, one or more of the electrical elements may form at least a portion of a transistor, such as a field-effect transistor, e.g., as is discussed in more detail herein. The electrical elements may be distributed within the device in any suitable configuration, for example, in an ordered array or randomly distributed. In some cases, the electrical elements are distributed such that an increasing concentration of electrical elements can be found towards the first portion of the device.

In some cases, some or all of the electrical elements are individually electrically addressable within the device. For instance, in some cases, at least about 10%, at least about 20%, at least about 30%, at least about 40%, at least about 50%, at least about 60%, at least about 70%, at least about 80%, at least about 90%, or substantially all of the electrical elements may be individually electrically addressable. In some embodiments, an electrical property of electrical elements can be individually determinable (e.g., being partially or fully resolvable without also including the electrical properties of other electrical elements), and/or such that the electrical property of an electrical element may be individually controlled (for example, by applying a desired voltage or current to the electrical element, for instance, without simultaneously applying the voltage or current to other electrical elements). In other embodiments, however, at least some of the electrical elements can be controlled within the same electronic circuit (e.g., by incorporating the electrical elements in series and/or in parallel), such that the electrical elements can still be electrically controlled and/or determined.

In various embodiments, more than one electrical element may be present within the device. The electrical elements may each independently be the same or different. For example, the device may comprise at least 5 electrical elements, at least about 10 electrical elements, at least about 15 electrical elements, at least about 20 electrical elements, at least about 25 electrical elements, at least about 30 electrical elements, at least about 50 electrical elements, at least about 100 electrical elements, at least about 300 electrical elements, at least about 1000 electrical elements, etc.

In addition, in some embodiments, there may be a relatively high density of electrical elements within the device, or at least a portion of the device. The electrical elements may be distributed uniformly or non-uniformly on the device or a portion thereof, e.g., a first portion. In some cases, the electrical elements may be distributed at an average density of at least about 5 elements/mm², at least about 10 elements/mm², at least about 30 elements/mm², at least about 50 elements/mm², at least about 75 elements/mm², at least about 100 elements/mm², at least about 300 elements/mm², at least about 500 elements/mm², at least about 750 elements/mm², at least about 1000 elements/mm², etc. In certain embodiments, the electrical elements are distributed such that the average separation between an electrical element and its nearest neighboring electrical element is less than about 2 mm, less than about 1 mm, less than about 500 micrometers, less than about 300 micrometers, less than about 100 micrometers, less than about 50 micrometers, less than about 30 micrometers, or less than about 10 micrometers.

Some or all of the electrical elements may be in electrical communication with one or more electrical contacts (e.g., in a second portion of the device) via one or more conductive pathways (e.g., passing through a joining portion, if present). The electrical contacts may be positioned on a second portion of the device that is not inserted into the tissue. The electrical contacts may be made out of any suitable material that allows transmission of an electrical signal. For example, the electrical contacts may comprise gold, silver, copper, aluminum, tantalum, titanium, nickel, tungsten, chromium, palladium, etc. In some cases, the electrical contacts have an average cross-section of less than about 10 micrometers, less than about 8 micrometers, less than about 6 micrometers, less than about 5 micrometers, less than about 4 micrometers, less than about 3 micrometers, less than about 2 micrometers, less than about 1 micrometer, etc.

In some embodiments, the electrical contacts can be used to determine a property of an electrical element within the device (for example, an electrical property or a chemical property as is discussed herein), and/or to direct an electrical signal to the electrical element, e.g., to electrically stimulate cells proximate the electrical element. The conductive pathways can form an electrical circuit that is internally contained within the device, and/or that extends externally of the device, e.g., such that the electrical circuit is in electrical communication with an external electrical system, such as a computer or a transmitter (for instance, a radio transmitter, a wireless transmitter, an Internet connection, etc.), as discussed herein. Any suitable conductive pathway may be used, for example, pathways comprising metals, semiconductors, conductive polymers, or the like.

Furthermore, more than one conductive pathway may be used in certain embodiments. For example, multiple conductive pathways can be used such that some or all of the electrical elements within the device may be electrically individually addressable. However, in other embodiments, more than one electrical element may be addressable by a particular conductive pathway. In addition, in some cases, other electric components may also be present within the device, e.g., as part of a conductive pathway or otherwise forming part of an electrical circuit. Examples include, but are not limited to, transistors such as field-effect transistors or bipolar junction transistors, resistors, capacitors, inductors, diodes, integrated circuits, etc. In certain cases, some of these may also comprise nanoscale wires and/or microscale wires. For example, in some embodiments, two sets of electrical contacts and conductive pathways, and an electrical element such as a nanoscale wire, may be used to define a transistor such as a field effect transistor, e.g., where the nanoscale wire or other electrical element defines the gate. As mentioned, the environment in and/or around the electrical element can affect the ability of the electrical element to function as a gate, and thus, the electrical element can be used as a sensor in some embodiments.

As mentioned, in various embodiments, one or more electrodes, electrical connectors, and/or conductive pathways may be positioned in electrical and/or physical communication with the electrical elements. These can be patterned to be in direct physical contact the electrical elements and/or there may be other materials that allow electrical communication to occur. Metals may be used due to their high conductance, e.g., such that changes within electrical properties obtained from the conductive pathway may be related to changes in properties of the electrical elements, rather than changes in properties of the conductive pathway. However, in other embodiments, other types of electrode materials are used, in addition or instead of metals.

A wide variety of metals may be used in various embodiments of the invention, for example in an electrode, electrical connector, conductive pathway, metal construct, polymer construct, etc. As non-limiting examples, the metals may include one or more of aluminum, gold, silver, copper, molybdenum, tantalum, titanium, nickel, tungsten, chromium, palladium, as well as any combinations of these and/or other metals. In some cases, the metal may be chosen to be one that is readily introduced, e.g., using techniques compatible with lithographic techniques. For example, in one set of embodiments, lithographic techniques such as e-beam lithography, photolithography, X-ray lithography, extreme ultraviolet lithography, ion projection lithography, etc. can be used to pattern or deposit one or more metals.

Additional processing steps can also be used to define or register the electrode, electrical connector, conductive pathway, metal construct, polymer construct, electrical elements, etc. in some cases, e.g., within the first portion, the second portion, and/or the joining portion. Thus, for example, the thickness of one of these may be less than about 1 mm, less than about 500 micrometers, less than about 300 micrometers, less than about 200 micrometers, less than about 100 micrometers, less than about 50 micrometers, less than about 30 micrometers, less than about 20 micrometers, less than about 10 micrometers, less than about 5 micrometers, less than about 4 micrometers, less than about 3 micrometers, less than about 2 micrometers, less than about 1 micrometer, less than about 700 nm, less than about 600 nm, less than about 500 nm, less than about 300 nm, less than about 200 nm, less than about 100 nm, less than about 80 nm, less than about 50 nm, less than about 30 nm, less than about 10 nm, less than about 5 nm, less than about 2 nm, etc. The thickness of the electrode may also be at least about 10 nm, at least about 20 nm, at least about 40 nm, at least about 60 nm, at least about 80 nm, or at least about 100 nm. For example, the thickness may be between about 40 nm and about 100 nm, between about 50 nm and about 80 nm.

In some embodiments, more than one metal may be used. The metals can be deposited in different regions or alloyed together, or in some cases, the metals may be layered on top of each other, e.g., layered on top of each other using various lithographic techniques. For example, a second metal may be deposited on a first metal, and in some cases, a third metal may be deposited on the second metal, etc. Additional layers of metal (e.g., fourth, fifth, sixth, etc.) can also be used in some embodiments. The metals may all be different, or in some cases, some of the metals (e.g., the first and third metals) may be the same. Each layer may independently be of any suitable thickness or dimension, e.g., of the dimensions described above, and the thicknesses of the various layers may independently be the same or different.

In some cases, the electrodes may include portions of metals and/or semiconductors, such as those described herein, that are not covered with an insulating material, such as a polymer. Such metals may be exposed to the external environment (for example, the subject once introduced into a subject), and accordingly, in some cases, such electrodes may be used to determine a physical property of a subject, and/or provide a stimulus (e.g., an electrical stimulus) to a subject. The electrode may include metals such as aluminum, gold, silver, copper, molybdenum, tantalum, titanium, nickel, tungsten, chromium, palladium, platinum, as well as any combinations of these and/or other metals, and/or semiconductor materials such as silicon, gallium, germanium, diamond (carbon), tin, selenium, tellurium, boron, phosphorous, and/or other semiconductors described herein (including elemental and compound semiconductors). The electrodes may include nanoscale wires and/or microscale wires in certain embodiments of the invention.

Thus, in some cases, the electrical elements may include nanoscale wires. Any nanoscale wire can be used in the device, e.g., as a nanoscale sensing element. Non-limiting examples of suitable nanoscale wires include carbon nanotubes, nanorods, nanowires, organic and inorganic conductive and semiconducting polymers, metal nanoscale wires, semiconductor nanoscale wires (for example, formed from silicon), and the like. If carbon nanotubes are used, they may be single-walled and/or multi-walled, and may be metallic and/or semiconducting in nature. Other conductive or semiconducting elements that may not be nanoscale wires, but are of various small nanoscopic-scale dimension, also can be used in certain embodiments. However, it should be understood that in some cases, larger electrical elements may also be used, e.g., microscale wires, in addition to or instead of nanoscale wires.

In general, a “nanoscale wire” (also known herein as a “nanoscopic-scale wire” or “nanoscopic wire”) generally is a wire or other nanoscale object, that at any point along its length, has at least one cross-sectional dimension and, in some embodiments, two orthogonal cross-sectional dimensions (e.g., a diameter) of less than 1 micrometer, less than about 500 nm, less than about 200 nm, less than about 150 nm, less than about 100 nm, less than about 70, less than about 50 nm, less than about 20 nm, less than about 10 nm, less than about 5 nm, than about 2 nm, or less than about 1 nm. It should be understood that in many of the embodiments described herein, microscale wires may be used, e.g., in addition to and/or instead of nanoscale wires. Similarly, a “microscale wire” is a wire or other microscale object that is larger than a nanoscale wire, and that at any point along its length, has at least one cross-sectional dimension and, in some embodiments, two orthogonal cross-sectional dimensions (e.g., a diameter) of less than 1 mm, less than 500 micrometers, less than about 200 micrometers, less than about 150 micrometers, less than about 100 micrometers, less than about 70, less than about 50 micrometers, less than about 20 micrometers, less than about 10 micrometers, less than about 5 micrometers, or than about 2 micrometers.

In some embodiments, the nanoscale or microscale wire is generally cylindrical. In other embodiments, however, other shapes are possible; for example, the nanoscale wire or microscale wire can be faceted, i.e., the nanoscale wire or microscale wire may have a polygonal cross-section. The cross-section of a nanoscale wire or microscale wire can be of any arbitrary shape, including, but not limited to, circular, square, rectangular, annular, polygonal, or elliptical, and may be a regular or an irregular shape. The nanoscale wire or microscale wire can also be solid or hollow.

In some cases, the nanoscale wire or microscale wire has one dimension that is substantially longer than the other dimensions of the nanoscale wire or microscale wire. For example, the nanoscale wire or microscale wire may have a longest dimension that is at least about 1 micrometer, at least about 3 micrometers, at least about 5 micrometers, or at least about 10 micrometers or about 20 micrometers in length, and/or the nanoscale wire or microscale wire may have an aspect ratio (longest dimension to shortest orthogonal dimension) of greater than about 2:1, greater than about 3:1, greater than about 4:1, greater than about 5:1, greater than about 10:1, greater than about 25:1, greater than about 50:1, greater than about 75:1, greater than about 100:1, greater than about 150:1, greater than about 250:1, greater than about 500:1, greater than about 750:1, or greater than about 1000:1 or more in some cases.

In some embodiments, a nanoscale wire or microscale wire may be substantially uniform, or have a variation in average diameter of the nanoscale wire or microscale wire of less than about 30%, less than about 25%, less than about 20%, less than about 15%, less than about 10%, or less than about 5%. In some embodiments, the nanoscale wire or microscale wire may be grown from substantially uniform nanoclusters or particles, e.g., colloid particles. See, e.g., U.S. Pat. No. 7,301,199, issued Nov. 27, 2007, entitled “Nanoscale Wires and Related Devices,” by Lieber, et al., incorporated herein by reference in its entirety. In some cases, the nanoscale wire or microscale wire may be one of a population of nanoscale wires or microscale wires having an average variation in diameter, of the population of nanoscale or microscale wires, of less than about 30%, less than about 25%, less than about 20%, less than about 15%, less than about 10%, or less than about 5%.

In some embodiments, a nanoscale wire or microscale wire has a conductivity of or of similar magnitude to any semiconductor or any metal. The nanoscale wire or microscale wire can be formed of suitable materials, e.g., semiconductors, metals, etc., as well as any suitable combinations thereof. In some cases, the nanoscale wire or microscale wire will have the ability to pass electrical charge, for example, being electrically conductive. For example, the nanoscale wire may have a relatively low resistivity, e.g., less than about 10⁻³ Ohm m, less than about 10⁻⁴ Ohm m, less than about 10⁻⁶ Ohm m, or less than about 10⁻⁷ Ohm m. The nanoscale wire or microscale wire can, in some embodiments, have a conductance of at least about 10 nanosiemens, at least about 30 nanosiemens, at least about 50 nanosiemens, at least about 100 nanosiemens, at least about 300 nanosiemens, at least about 500 nanosiemens, at least about 1 microsiemens, at least about 3 microsiemens, at least about 10 microsiemens, at least about 30 microsiemens, or at least about 100 microsiemens.

The nanoscale wire or microscale wire can be solid or hollow, in various embodiments. As used herein, a “nanotube” (or a “microtube”) is a nanoscale wire (or a microscale wire) that is hollow, or that has a hollowed-out core, including those nanotubes or microtubes known to those of ordinary skill in the art. As another example, a nanotube or microtube may be created by creating a core/shell nanowire or microwire, then etching away at least a portion of the core to leave behind a hollow shell. In one set of embodiments, the nanoscale wire is a non-carbon nanotube. In contrast, a “nanowire” (or a “microwire”) is a nanoscale wire (or a microscale wire) that is typically solid (i.e., not hollow). Thus, for example, a nanoscale wire may be a semiconductor nanowire, such as a silicon nanowire.

In one set of embodiment, a nanoscale wire or microscale wire may comprise or consist essentially of a metal. Non-limiting examples of potentially suitable metals include aluminum, gold, silver, copper, molybdenum, tantalum, titanium, nickel, tungsten, chromium, or palladium.

In another set of embodiments, a nanoscale wire or microscale wire comprises or consists essentially of a semiconductor. Typically, a semiconductor is an element having semiconductive or semi-metallic properties (i.e., between metallic and non-metallic properties). An example of a semiconductor is silicon. Other non-limiting examples include elemental semiconductors, such as gallium, germanium, diamond (carbon), tin, selenium, tellurium, boron, or phosphorous. In other embodiments, more than one element may be present in the nanoscale wire as the semiconductor, for example, gallium arsenide, gallium nitride, indium phosphide, cadmium selenide, etc. Still other examples include a Group II-VI material (which includes at least one member from Group II of the Periodic Table and at least one member from Group VI, for example, ZnS, ZnSe, ZnSSe, ZnCdS, CdS, or CdSe), or a Group III-V material (which includes at least one member from Group III and at least one member from Group V, for example GaAs, GaP, GaAsP, InAs, InP, AlGaAs, or InAsP). In some cases, at least one of the nanoscale wires is a silicon nanowire.

In certain embodiments, the semiconductor can be undoped or doped (e.g., p-type or n-type). For example, in one set of embodiments, a nanoscale wire or a microscale wire may be a p-type semiconductor nanoscale wire or an n-type semiconductor wire, and can be used as a component of a transistor such as a field effect transistor (“FET”). For instance, the nanoscale wire or microscale wire may act as the “gate” of a source-gate-drain arrangement of a FET, while metal leads or other conductive pathways (as discussed herein) are used as the source and drain electrodes.

In some embodiments, a dopant or a semiconductor may include mixtures of Group IV elements, for example, a mixture of silicon and carbon, or a mixture of silicon and germanium. In other embodiments, the dopant or the semiconductor may include a mixture of a Group III and a Group V element, for example, BN, BP, BAs, AN, AlP, AlAs, AlSb, GaN, GaP, GaAs, GaSb, InN, InP, InAs, or InSb. Mixtures of these may also be used, for example, a mixture of BN/BP/BAs, or BN/AlP. In other embodiments, the dopants may include alloys of Group III and Group V elements. For example, the alloys may include a mixture of AlGaN, GaPAs, InPAs, GaInN, AlGaInN, GaInAsP, or the like. In other embodiments, the dopants may also include a mixture of Group II and Group VI semiconductors. For example, the semiconductor may include ZnO, ZnS, ZnSe, ZnTe, CdS, CdSe, CdTe, HgS, HgSe, HgTe, BeS, BeSe, BeTe, MgS, MgSe, or the like. Alloys or mixtures of these dopants are also possible, for example, (ZnCd)Se, or Zn(SSe), or the like. Additionally, alloys of different groups of semiconductors may also be possible, for example, a combination of a Group II-Group VI and a Group III-Group V semiconductor, for example, (GaAs)ₓ(ZnS)₁₋ₓ. Other examples of dopants may include combinations of Group IV and Group VI elements, such as GeS, GeSe, GeTe, SnS, SnSe, SnTe, PbO, PbS, PbSe, or PbTe. Other semiconductor mixtures may include a combination of a Group I and a Group VII, such as CuF, CuCl, CuBr, CuI, AgF, AgCl, AgBr, AgI, or the like. Other dopant compounds may include different mixtures of these elements, such as BeSiN₂, CaCN₂, ZnGeP₂, CdSnAs₂, ZnSnSb₂, CuGeP₃, CuSi₂P₃, Si₃N₄, Ge₃N₄, Al₂O₃, (Al, Ga, In)₂(S, Se, Te)₃, Al₂CO, (Cu, Ag)(Al, Ga, In, Tl, Fe)(S, Se, Te)₂ and the like.

The doping of the semiconductor to produce a p-type or n-type semiconductor may be achieved via bulk-doping in certain embodiments, although in other embodiments, other doping techniques (such as ion implantation) can be used. Many such doping techniques that can be used will be familiar to those of ordinary skill in the art, including both bulk doping and surface doping techniques. A bulk-doped article (e.g. an article, or a section or region of an article) is an article for which a dopant is incorporated substantially throughout the crystalline lattice of the article, as opposed to an article in which a dopant is only incorporated in particular regions of the crystal lattice at the atomic scale, for example, only on the surface or exterior. For example, some articles are typically doped after the base material is grown, and thus the dopant only extends a finite distance from the surface or exterior into the interior of the crystalline lattice. It should be understood that “bulk-doped” does not define or reflect a concentration or amount of doping in a semiconductor, nor does it necessarily indicate that the doping is uniform. “Heavily doped” and “lightly doped” are terms the meanings of which are clearly understood by those of ordinary skill in the art. In some embodiments, one or more regions comprise a single monolayer of atoms (“delta-doping”). In certain cases, the region may be less than a single monolayer thick (for example, if some of the atoms within the monolayer are absent). As a specific example, the regions may be arranged in a layered structure within the nanoscale wire, and one or more of the regions can be delta-doped or partially delta-doped.

Accordingly, in one set of embodiments, the nanoscale wire or microscale wire may include a heterojunction, e.g., of two regions with dissimilar materials or elements, and/or the same materials or elements but at different ratios or concentrations. The regions of the wire may be distinct from each other with minimal cross-contamination, or the composition of the nanoscale wire can vary gradually from one region to the next. The regions may be both longitudinally arranged relative to each other, or radially arranged (e.g., as in a core/shell arrangement) on the wire. Each region may be of any size or shape within the wire. The junctions may be, for example, a p/n junction, a p/p junction, an n/n junction, a p/i junction (where i refers to an intrinsic semiconductor), an n/i junction, an i/i junction, or the like. The junction can also be a Schottky junction in some embodiments. The junction may also be, for example, a semiconductor/semiconductor junction, a semiconductor/metal junction, a semiconductor/insulator junction, a metal/metal junction, a metal/insulator junction, an insulator/insulator junction, or the like. The junction may also be a junction of two materials, a doped semiconductor to a doped or an undoped semiconductor, or a junction between regions having different dopant concentrations. The junction can also be a defected region to a perfect single crystal, an amorphous region to a crystal, a crystal to another crystal, an amorphous region to another amorphous region, a defected region to another defected region, an amorphous region to a defected region, or the like. More than two regions may be present, and these regions may have unique compositions or may comprise the same compositions. As one example, a wire can have a first region having a first composition, a second region having a second composition, and a third region having a third composition or the same composition as the first composition. Non-limiting examples of nanoscale wires comprising heterojunctions (including core/shell heterojunctions, longitudinal heterojunctions, etc., as well as combinations thereof) are discussed in U.S. Pat. No. 7,301,199, issued Nov. 27, 2007, entitled “Nanoscale Wires and Related Devices,” by Lieber, et al., incorporated herein by reference in its entirety.

In some embodiments, the nanoscale wire or microscale wire is bent or kinked. A kink is typically a relatively sharp transition or turning between a first substantially straight portion of a wire and a second substantially straight portion of a wire. For example, a wire may have 1, 2, 3, 4, or 5 or more kinks. In some cases, the wire is formed from a single crystal and/or comprises or consists essentially of a single crystallographic orientation, for example, a <110> crystallographic orientation, a <112> crystallographic orientation, or a <1120> crystallographic orientation. It should be noted that the kinked region need not have the same crystallographic orientation as the rest of the wire. In some embodiments, a kink in the wire may be at an angle of about 120° or a multiple thereof. The kinks can be intentionally positioned along the wire in some cases. For example, a wire may be grown from a catalyst particle by exposing the catalyst particle to various gaseous reactants to cause the formation of one or more kinks within the nanoscale wire. Non-limiting examples of kinked wires, and suitable techniques for making such wires, are disclosed in International Patent Application No. PCT/US2010/050199, filed Sep. 24, 2010, entitled “Bent Nanowires and Related Probing of Species,” by Tian, et al., published as WO 2011/038228 on Mar. 31, 2011, incorporated herein by reference in its entirety.

In one set of embodiments, the nanoscale wire or microscale wire is formed from a single crystal, for example, a single crystal nanoscale wire comprising a semiconductor. A single crystal item may be formed via covalent bonding, ionic bonding, or the like, and/or combinations thereof. While such a single crystal item may include defects in the crystal in some cases, the single crystal item is distinguished from an item that includes one or more crystals, not ionically or covalently bonded, but merely in close proximity to one another.

In some embodiments, the nanoscale wires or microscale wires used herein are individual or free-standing nanoscale wires. For example, an “individual” or a “free-standing” wire may, at some point in its life, not be attached to another article, for example, with another wire, or the free-standing wire may be in solution. This is in contrast to nanoscale features etched onto the surface of a substrate, e.g., a silicon wafer, in which the nanoscale features are never removed from the surface of the substrate as a free-standing article. This is also in contrast to conductive portions of articles which differ from surrounding material only by having been altered chemically or physically, in situ, i.e., where a portion of a uniform article is made different from its surroundings by selective doping, etching, etc. An “individual” or a “free-standing” wire is one that can be (but need not be) removed from the location where it is made, as an individual article, and transported to a different location and combined with different components to make a functional device such as those described herein.

The nanoscale wire or microscale wire, in some embodiments, may be a sensing element responsive to a property external of the wire, e.g., a chemical property, an electrical property, a physical property, etc. Such determination may be qualitative and/or quantitative, and such determinations may also be recorded, e.g., for later use. For example, in one set of embodiments, the wire may be responsive to voltage. For instance, the nanoscale wire or microscale wire may exhibit a voltage sensitivity of at least about 5 microsiemens/V; by determining the conductivity of a nanoscale wire, the voltage surrounding the wire may thus be determined. In other embodiments, the voltage sensitivity can be at least about 10 nanosiemens/V, at least about 30 nanosiemens/V, at least about 50 nanosiemens/V, at least about 100 nanosiemens/V, at least about 300 nanosiemens/V, at least about 500 nanosiemens/V, at least about 1 microsiemens/V, at least about 3 microsiemens/V, at least about 5 microsiemens/V, at least about 10 microsiemens/V, at least about 30 microsiemens/V, at least about 50 microsiemens/V, or at least about 100 microsiemens/V. Other examples of electrical properties that can be determined include resistance, resistivity, conductance, conductivity, impendence, or the like.

As another example, a nanoscale wire or microscale wire may be a sensing element responsive to a chemical property of the environment surrounding the wire. For example, an electrical property of the wire can be affected by a chemical environment surrounding the wire, and the electrical property can be thereby determined to determine the chemical environment surrounding the nanoscale wire. As a specific non-limiting example, a nanoscale wire or microscale wire may be sensitive to pH or hydrogen ions. Further non-limiting examples of such wires are discussed in U.S. Pat. No. 7,129,554, filed Oct. 31, 2006, entitled “Nanosensors,” by Lieber, et al., incorporated herein by reference in its entirety.

As a non-limiting example, the nanoscale wire or microscale wire may be a sensing element having the ability to bind to an analyte indicative of a chemical property of the environment surrounding the nanoscale wire or microscale wire (e.g., hydrogen ions for pH, or concentration for an analyte of interest), and/or the wire may be partially or fully functionalized, i.e. comprising surface functional moieties, to which an analyte is able to bind, thereby causing a determinable property change to the nanoscale wire or microscale wire, e.g., a change to the resistivity or impedance of the wire. The binding of the analyte can be specific or non-specific. Functional moieties may include simple groups, selected from the groups including, but not limited to, —OH, —CHO, —COOH, —SO₃H, —CN, —NH₂, —SH, —COSH, —COOR, halide; biomolecular entities including, but not limited to, amino acids, proteins, sugars, DNA, antibodies, antigens, and enzymes; grafted polymer chains with chain length less than the diameter of the wire, selected from a group of polymers including, but not limited to, polyamide, polyester, polyimide, polyacrylic; a shell of material comprising, for example, metals, semiconductors, and insulators, which may be a metallic element, an oxide, an sulfide, a nitride, a selenide, a polymer and a polymer gel. A non-limiting example of a protein is PSA (prostate specific antigen), which can be determined, for example, by modifying the wires by binding monoclonal antibodies for PSA (Abl) thereto. See, e.g., U.S. Pat. No. 8,232,584, issued Jul. 31, 2012, entitled “Nanoscale Sensors,” by Lieber, et al., incorporated herein by reference in its entirety.

In some embodiments, a reaction entity may be bound to a surface of the nanoscale wire or microscale wire, and/or positioned in relation to the wire such that the analyte can be determined by determining a change in a property of the nanoscale wire or microscale wire, e.g., acting as a sensing element. The “determination” may be quantitative and/or qualitative, depending on the application, and in some cases, the determination may also be analyzed, recorded for later use, transmitted, or the like. The term “reaction entity” refers to any entity that can interact with an analyte in such a manner to cause a detectable change in a property (such as an electrical property) of a nanoscale wire or microscale wire. The reaction entity may enhance the interaction between the wire and the analyte, or generate a new chemical species that has a higher affinity to the wire, or to enrich the analyte around the wire. The reaction entity can comprise a binding partner to which the analyte binds. The reaction entity, when a binding partner, can comprise a specific binding partner of the analyte. For example, the reaction entity may be a nucleic acid, an antibody, a sugar, a carbohydrate or a protein. Alternatively, the reaction entity may be a polymer, catalyst, or a quantum dot. A reaction entity that is a catalyst can catalyze a reaction involving the analyte, resulting in a product that causes a detectable change in the nanowire, e.g. via binding to an auxiliary binding partner of the product electrically coupled to the nanowire. Another exemplary reaction entity is a reactant that reacts with the analyte, producing a product that can cause a detectable change in the wire. The reaction entity can comprise a shell on the wire, e.g. a shell of a polymer that recognizes molecules in, e.g., a gaseous sample, causing a change in conductivity of the polymer which, in turn, causes a detectable change in the nanowire.

The term “binding partner” refers to a molecule that can undergo binding with a particular analyte, or “binding partner” thereof, and includes specific, semi-specific, and non-specific binding partners as known to those of ordinary skill in the art. The term “specifically binds,” when referring to a binding partner (e.g., protein, nucleic acid, antibody, etc.), refers to a reaction that is determinative of the presence and/or identity of one or other member of the binding pair in a mixture of heterogeneous molecules (e.g., proteins and other biologics). Thus, for example, in the case of a receptor/ligand binding pair the ligand would specifically and/or preferentially select its receptor from a complex mixture of molecules, or vice versa. An enzyme would specifically bind to its substrate, a nucleic acid would specifically bind to its complement, an antibody would specifically bind to its antigen. Other examples include, nucleic acids that specifically bind (hybridize) to their complement, antibodies specifically bind to their antigen, and the like. The binding may be by one or more of a variety of mechanisms including, but not limited to ionic interactions, and/or covalent interactions, and/or hydrophobic interactions, and/or van der Waals interactions, etc.

The antibody may be any protein or glycoprotein comprising or consisting essentially of one or more polypeptides substantially encoded by immunoglobulin genes or fragments of immunoglobulin genes. Examples of recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist as intact immunoglobulins or as a number of well characterized fragments produced by digestion with various peptidases. Thus, for example, pepsin digests an antibody below (i.e. toward the Fc domain) the disulfide linkages in the hinge region to produce F(ab)′₂, a dimer of Fab which itself is a light chain joined to VHCH1 by a disulfide bond. The F(ab)′₂ may be reduced under mild conditions to break the disulfide linkage in the hinge region thereby converting the (Fab)₂ dimer into an Fab′ monomer. The Fab′ monomer is essentially a Fab with part of the hinge region. While various antibody fragments are defined in terms of the digestion of an intact antibody, one of skill will appreciate that such fragments may be synthesized de novo either chemically, by utilizing recombinant DNA methodology, or by “phage display” methods. Non-limiting examples of antibodies include single chain antibodies, e.g., single chain Fv (scFv) antibodies in which a variable heavy and a variable light chain are joined together (directly or through a peptide linker) to form a continuous polypeptide.

Thus, in some embodiments, a property such as a chemical property and/or an electrical property can be determined, e.g., at a resolution of less than about 2 mm, less than about 1 mm, less than about 500 micrometers, less than about 300 micrometers, less than about 100 micrometers, less than about 50 micrometers, less than about 30 micrometers, or less than about 10 micrometers, etc., e.g., due to the average separation between an electrical element (such as a nanoscale wire) and its nearest neighboring electrical element. In addition, the property may be determined within the tissue in 3 dimensions in some instances, in contrast with many other techniques where only a surface of the biological tissue can be studied. Accordingly, very high resolution and/or 3-dimensional mappings of the property of the biological tissue can be obtained in some embodiments. Any suitable tissue may be studied, e.g., brain tissue, eyes (e.g., the retina), the spinal cord or other nerves, cardiac tissue, vascular tissue, muscle, cartilage, bone, liver tissue, pancreatic tissue, bladder tissue, airway tissues, bone marrow tissue, or the like.

In addition, in some cases, such properties can be determined and/or recorded as a function of time. Thus, for example, such properties can be determined at a time resolution of less than about 1 min, less than about 30 s, less than about 15 s, less than about 10 s, less than about 5 s, less than about 3 s, less than about 1 s, less than about 500 ms, less than about 300 ms, less than about 100 ms, less than about 50 ms, less than about 30 ms, less than about 10 ms, less than about 5 ms, less than about 3 ms, less than about 1 ms, etc.

In yet another set of embodiments, the biological tissue, and/or portions of the biological tissue, may be electrically stimulated using nanoscale wires and/or microscale wires present within the tissue. For example, all, or a subset of the electrical elements may be electrically stimulated, e.g., by using an external electrical system, such as a computer, for example, as previously discussed. Thus, for example, a single electrical element, a group of electrical elements, or substantially all of the electrical elements can be electrically stimulated, depending on the particular application. In some cases, such electrical elements can be stimulated in a particular pattern, e.g., to cause cardiac or muscle cells to contract or beat in a particular pattern (for example, as part of a prosthetic or a pacemaker), to cause the firing of neurons with a particular pattern, to monitor the status of an implanted tissue within a living subject, or the like.

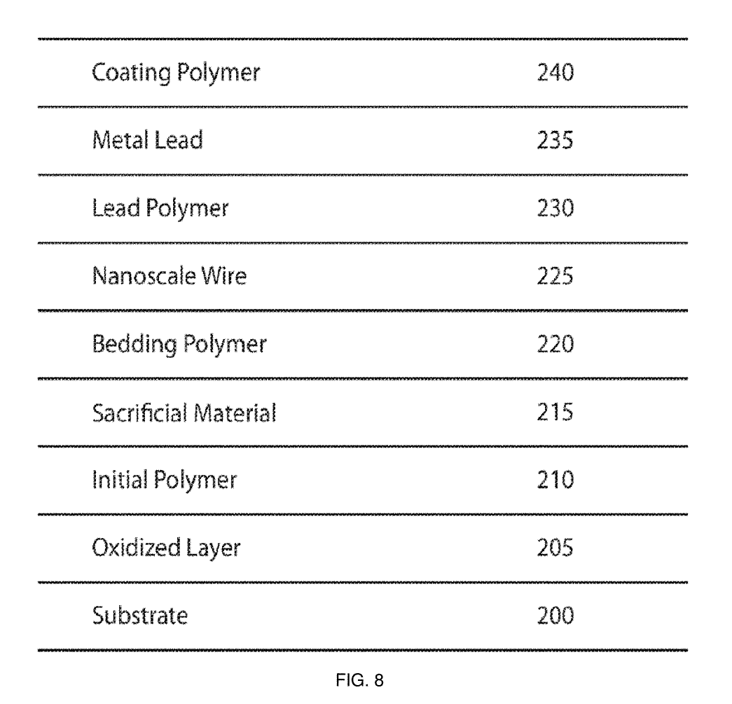

Another aspect of the present invention is generally directed to systems and methods for making and using such devices, e.g., for insertion into matter. Briefly, in one set of embodiments, a device can be constructed by assembling various polymers, metals, nanoscale wires, microscale wires, and/or other components together on a substrate. Portions of the device (e.g., a first portion, a second portion, and/or a joining portion, if present) may be fabricated using the same or different techniques, including any of the ones discussed herein, and thus may include the same or different materials. For example, lithographic techniques such as e-beam lithography, photolithography, X-ray lithography, extreme ultraviolet lithography, ion projection lithography, etc. may be used to pattern polymers, metals, etc. on the substrate, and nanoscale wires and/or microscale wires can be prepared separately then added to the substrate. After assembly, at least a portion of the substrate (e.g., a sacrificial material) may be removed, allowing the device to be partially or completely removed from the substrate. The device can, in some cases, be formed into a 3-dimensional structure, for example, spontaneously, or by folding or rolling the structure. Other materials may also be added to the device, e.g., to help stabilize the structure, to add additional agents to enhance its biocompatibility, etc. The device can be used in vivo, e.g., by implanting it in a living subject, and/or in vitro, e.g., by seeding cells, etc. on the device. In addition, in some cases, cells may initially be grown on the device before the device is implanted into a subject. A schematic diagram of the layers formed on the substrate in one embodiment is shown in FIG. 8. However, it should be understood that this diagram is illustrative only and is not drawn to scale, and not all of the layers shown in FIG. 8 are necessarily required in every embodiment of the invention. In addition, it should be understood that in other embodiments, the device can be used in non-living subjects.

The substrate (200 in FIG. 8) may be chosen to be one that can be used for lithographic techniques such as e-beam lithography or photolithography, or other lithographic techniques including those discussed herein. For example, the substrate may comprise or consist essentially of a semiconductor material such as silicon, although other substrate materials (e.g., a metal) can also be used. Typically, the substrate is one that is substantially planar, e.g., so that polymers, metals, and the like can be patterned on the substrate.

In some cases, a portion of the substrate can be oxidized, e.g., forming SiO₂ and/or Si₃N₄ on a portion of the substrate, which may facilitate subsequent addition of materials (metals, polymers, etc.) to the substrate. In some cases, the oxidized portion may form a layer of material on the substrate (205 in FIG. 8), e.g., having a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc.

In certain embodiments, one or more polymers can also be deposited or otherwise formed prior to depositing the sacrificial material. In some cases, the polymers may be deposited or otherwise formed as a layer of material (210 in FIG. 8) on the substrate. Deposition may be performed using any suitable technique, e.g., using lithographic techniques such as e-beam lithography, photolithography, X-ray lithography, extreme ultraviolet lithography, ion projection lithography, etc. In some cases, some or all of the polymers may be biocompatible and/or biodegradable. The polymers that are deposited may also comprise methyl methacrylate and/or poly(methyl methacrylate), in some embodiments. One, two, or more layers of polymer can be deposited (e.g., sequentially) in various embodiments, and each layer may independently have a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc.

Next, a sacrificial material may be deposited. The sacrificial material can be chosen to be one that can be removed without substantially altering other materials (e.g., polymers, other metals, nanoscale wires, microscale wires, etc.) deposited thereon. For example, in one embodiment, the sacrificial material may be a metal, e.g., one that is easily etchable. For instance, the sacrificial material can comprise germanium or nickel, which can be etched or otherwise removed, for example, using a peroxide (e.g., H₂O₂) or a nickel etchant (many of which are readily available commercially). In some cases, the sacrificial material may be deposited on oxidized portions or polymers previously deposited on the substrate. In some cases, the sacrificial material is deposited as a layer (e.g., 215 in FIG. 8). The layer can have a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc.

In some embodiments, a “bedding” polymer can be deposited, e.g., on the sacrificial material. The bedding polymer may include one or more polymers, which may be deposited as one or more layers (220 in FIG. 8). The bedding polymer can be used to support the nanoscale wires and/or microscale wires, and in some cases, partially or completely surround the nanoscale wires and/or microscale wires, depending on the application. For example, as discussed below, one or more nanoscale wires and/or microscale wires may be deposited on at least a portion of the uppermost layer of bedding polymer.

For instance, the bedding polymer can at least partially define a device. In one set of embodiments, the bedding polymer may be deposited as a layer of material, such that portions of the bedding polymer may be subsequently removed. For example, the bedding polymer can be deposited using lithographic techniques such as e-beam lithography, photolithography, X-ray lithography, extreme ultraviolet lithography, ion projection lithography, etc., or using other techniques for removing polymer that are known to those of ordinary skill in the art. In some cases, more than one bedding polymer is used, e.g., deposited as more than one layer (e.g., sequentially), and each layer may independently have a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc. For example, in some embodiments, portions of the photoresist may be exposed to light (visible, UV, etc.), electrons, ions, X-rays, etc. (e.g., projected onto the photoresist), and the exposed portions can be etched away (e.g., using suitable etchants, plasma, etc.) to produce the pattern.

Accordingly, the bedding polymer may be formed into a particular pattern, e.g., in a grid, or in a pattern that suggests an endogenous probe, before or after deposition of nanoscale wires and/or microscale wires (as discussed in detail below), in certain embodiments of the invention. The pattern can be regular or irregular. For example, the bedding polymer can be formed into a pattern defining pore sizes such as those discussed herein. For instance, the polymer may have an average pore size of at least about 100 micrometers, at least about 200 micrometers, at least about 300 micrometers, at least about 400 micrometers, at least about 500 micrometers, at least about 600 micrometers, at least about 700 micrometers, at least about 800 micrometers, at least about 900 micrometers, or at least about 1 mm, and/or an average pore size of no more than about 1.5 mm, no more than about 1.4 mm, no more than about 1.3 mm, no more than about 1.2 mm, no more than about 1.1 mm, no more than about 1 mm, no more than about 900 micrometers, no more than about 800 micrometers, no more than about 700 micrometers, no more than about 600 micrometers, or no more than about 500 micrometers, etc.

Any suitable polymer may be used as the bedding polymer. In some cases, one or more of the polymers can be chosen to be biocompatible and/or biodegradable. In certain embodiments, one or more of the bedding polymers may comprise a photoresist. Photoresists can be useful due to their familiarity in use in lithographic techniques such as those discussed herein. Non-limiting examples of photoresists include SU-8, S1805, LOR 3A, poly(methyl methacrylate), poly(methyl glutarimide), phenol formaldehyde resin (diazonaphthoquinone/novolac), diazonaphthoquinone (DNQ), Hoechst AZ 4620, Hoechst AZ 4562, Shipley 1400-17, Shipley 1400-27, Shipley 1400-37, etc., as well as any others discussed herein.

In certain embodiments, one or more of the bedding polymers can be heated or baked, e.g., before or after depositing nanoscale wires and/or microscale wires thereon as discussed below, and/or before or after patterning the bedding polymer. For example, such heating or baking, in some cases, is important to prepare the polymer for lithographic patterning. In various embodiments, the bedding polymer may be heated to a temperature of at least about 30° C., at least about 65° C., at least about 95° C., at least about 150° C., or at least about 180° C., etc.

Next, one or more nanoscale wires and/or microscale wires (e.g., 225 in FIG. 8) may be deposited, e.g., on a bedding polymer on the substrate. Any of the nanoscale wires and/or microscale wires described herein may be used, e.g., n-type and/or p-type wires, substantially uniform wires (e.g., having a variation in average diameter of less than 20%), nanoscale wires having a diameter of less than about 1 micrometer, semiconductor wires, silicon nanowires, bent wires, kinked wires, core/shell wires, nanoscale or microscale wires with heterojunctions, etc. In some cases, the nanoscale wires and/or microscale wires are present in a liquid which is applied to the substrate, e.g., poured, painted, or otherwise deposited thereon. In some embodiments, the liquid is chosen to be relatively volatile, such that some or all of the liquid can be removed by allowing it to substantially evaporate, thereby depositing the nanoscale wires and/or microscale wires. In some cases, at least a portion of the liquid can be dried off, e.g., by applying heat to the liquid. Examples of suitable liquids include water or isopropanol.

In some cases, at least some of the nanoscale wires and/or microscale wires may be at least partially aligned, e.g., as part of the deposition process, and/or after the nanoscale wires and/or microscale wires have been deposited on the substrate. Thus, the alignment can occur before or after drying or other removal of the liquid, if a liquid is used. Any suitable technique may be used for alignment of the nanoscale wires and/or microscale wires. For example, the nanoscale wires and/or microscale wires can be aligned by passing or sliding substrates containing the nanoscale wires and/or microscale wires past each other (see, e.g., International Patent Application No. PCT/US2007/008540, filed Apr. 6, 2007, entitled “Nanoscale Wire Methods and Devices,” by Nam, et al., published as WO 2007/145701 on Dec. 21, 2007, incorporated herein by reference in its entirety), the nanoscale wires and/or microscale wires can be aligned using Langmuir-Blodgett techniques (see, e.g., U.S. patent application Ser. No. 10/995,075, filed Nov. 22, 2004, entitled “Nanoscale Arrays and Related Devices,” by Whang, et al., published as U.S. Patent Application Publication No. 2005/0253137 on Nov. 17, 2005, incorporated herein by reference in its entirety), the nanoscale wires and/or microscale wires can be aligned by incorporating the nanoscale wires and/or microscale wires in a liquid film or “bubble” which is deposited on the substrate (see, e.g., U.S. patent application Ser. No. 12/311,667, filed Apr. 8, 2009, entitled “Liquid Films Containing Nanostructured Materials,” by Lieber, et al., published as U.S. Patent Application Publication No. 2010/0143582 on Jun. 10, 2010, incorporated by reference herein in its entirety), or a gas or liquid can be passed across the nanoscale wires and/or microscale wires to align the nanoscale wires and/or microscale wires (see, e.g., U.S. Pat. No. 7,211,464, issued May 1, 2007, entitled “Doped Elongated Semiconductors, Growing Such Semiconductors, Devices Including Such Semiconductors, and Fabricating Such Devices,” by Lieber, et al.; and U.S. Pat. No. 7,301,199, issued Nov. 27, 2007, entitled “Nanoscale Wires and Related Devices,” by Lieber, et al., each incorporated herein by reference in its entirety). Combinations of these and/or other techniques can also be used in certain instances. In some cases, the gas may comprise an inert gas and/or a noble gas, such as nitrogen or argon.

In certain embodiments, a “lead” polymer is deposited (230 in FIG. 8), e.g., on the sacrificial material and/or on at least some of the nanoscale wires and/or microscale wires. The lead polymer may include one or more polymers, which may be deposited as one or more layers.

The lead polymer can be used to cover or protect metal leads or other conductive pathways, which may be subsequently deposited on the lead polymer. In some embodiments, the lead polymer can be deposited, e.g., as a layer of material such that portions of the lead polymer can be subsequently removed, for instance, using lithographic techniques such as e-beam lithography, photolithography, X-ray lithography, extreme ultraviolet lithography, ion projection lithography, etc., or using other techniques for removing polymer that are known to those of ordinary skill in the art, similar to the bedding polymers previously discussed. However, the lead polymers need not be the same as the bedding polymers (although they can be), and they need not be deposited using the same techniques (although they can be). In some cases, more than one lead polymer may be used, e.g., deposited as more than one layer (for example, sequentially), and each layer may independently have a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc.

Any suitable polymer can be used as the lead polymer. In some cases, one or more of the polymers may be chosen to be biocompatible and/or biodegradable. For example, in one set of embodiments, one or more of the polymers may comprise poly(methyl methacrylate). In certain embodiments, one or more of the lead polymers comprises a photoresist, such as those described herein.

In certain embodiments, one or more of the lead polymers may be heated or baked, e.g., before or after depositing nanoscale wires and/or microscale wires thereon as discussed below, and/or before or after patterning the lead polymer. For example, such heating or baking, in some cases, is important to prepare the polymer for lithographic patterning. In various embodiments, the lead polymer may be heated to a temperature of at least about 30° C., at least about 65° C., at least about 95° C., at least about 150° C., or at least about 180° C., etc.

Next, a metal or other conductive material can be deposited (235 in FIG. 8), e.g., on one or more of the lead polymer, the sacrificial material, the nanoscale wires and/or microscale wires, etc. to form a metal lead or other conductive pathway. More than one metal can be used, which may be deposited as one or more layers. For example, a first metal may be deposited, e.g., on one or more of the lead polymers, and a second metal may be deposited on at least a portion of the first metal. Optionally, more metals can be used, e.g., a third metal may be deposited on at least a portion of the second metal, and the third metal may be the same or different from the first metal. In some cases, each metal may independently have a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, less than about 80 nm, less than about 60 nm, less than about 40 nm, less than about 30 nm, less than about 20 nm, less than about 10 nm, less than about 8 nm, less than about 6 nm, less than about 4 nm, or less than about 2 nm, etc., and the layers may be of the same or different thicknesses.

Any suitable technique can be used for depositing metals, and if more than one metal is used, the techniques for depositing each of the metals may independently be the same or different. For example, in one set of embodiments, deposition techniques such as sputtering can be used. Other examples include, but are not limited to, physical vapor deposition, vacuum deposition, chemical vapor deposition, cathodic arc deposition, evaporative deposition, e-beam PVD, pulsed laser deposition, ion-beam sputtering, reactive sputtering, ion-assisted deposition, high-target-utilization sputtering, high-power impulse magnetron sputtering, gas flow sputtering, or the like.

The metals can be chosen in some cases such that the deposition process yields a pre-stressed arrangement, e.g., due to atomic lattice mismatch, which causes the subsequent metal leads to warp or bend, for example, once released from the substrate. Although such processes were typically undesired in the prior art, in certain embodiments of the present invention, such pre-stressed arrangements may be used to cause the resulting device to form a 3-dimensional structure, in some cases spontaneously, upon release from the substrate. However, it should be understood that in other embodiments, the metals may not necessary be deposited in a pre-stressed arrangement.

Examples of metals that can be deposited (stressed or unstressed) include, but are not limited to, aluminum, gold, silver, copper, molybdenum, tantalum, titanium, nickel, tungsten, chromium, palladium, as well as any combinations of these and/or other metals. For example, a chromium/palladium/chromium deposition process, in some embodiments, may form a pre-stressed arrangement that is able to spontaneously form a 3-dimensional structure after release from the substrate.

In certain embodiments, a “coating” polymer can be deposited (240 in FIG. 8), e.g., on at least some of the conductive pathways and/or at least some of the nanoscale wires and/or microscale wires. The coating polymer may include one or more polymers, which may be deposited as one or more layers. In some embodiments, the coating polymer may be deposited on one or more portions of a substrate, e.g., as a layer of material such that portions of the coating polymer can be subsequently removed, e.g., using lithographic techniques such as e-beam lithography, photolithography, X-ray lithography, extreme ultraviolet lithography, ion projection lithography, etc., or using other techniques for removing polymer that are known to those of ordinary skill in the art, similar to the other polymers previously discussed. The coating polymers can be the same or different from the lead polymers and/or the bedding polymers. In some cases, more than one coating polymer may be used, e.g., deposited as more than one layer (e.g., sequentially), and each layer may independently have a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc.

Any suitable polymer may be used as the coating polymer. In some cases, one or more of the polymers can be chosen to be biocompatible and/or biodegradable. For example, in one set of embodiments, one or more of the polymers may comprise poly(methyl methacrylate). In certain embodiments, one or more of the coating polymers may comprise a photoresist, e.g., as discussed herein.

In certain embodiments, one or more of the coating polymers can be heated or baked, e.g., before or after depositing nanoscale wires and/or microscale wires thereon as discussed below, and/or before or after patterning the coating polymer. For example, such heating or baking, in some cases, is important to prepare the polymer for lithographic patterning. In various embodiments, the coating polymer may be heated to a temperature of at least about 30° C., at least about 65° C., at least about 95° C., at least about 150° C., or at least about 180° C., etc.

After formation of the device, some or all of the sacrificial material may then be removed in some cases. In one set of embodiments, for example, at least a portion of the sacrificial material is exposed to an etchant able to remove the sacrificial material. For example, if the sacrificial material is a metal such as nickel, a suitable etchant (for example, a metal etchant such as a nickel etchant, etc.) can be used to remove the sacrificial metal. Many such etchants may be readily obtained commercially. In addition, in some embodiments, the device can also be dried, e.g., in air (e.g., passively), by using a heat source, by using a critical point dryer, etc. In certain embodiments, upon removal of the sacrificial material, pre-stressed portions of the device (e.g., metal leads containing dissimilar metals) can spontaneously cause the device to adopt a 3-dimensional structure. In some cases, the device may form a 3-dimensional structure as discussed herein. For example, the device may have an open porosity of at least about 30%, at least about 40%, at least about 50%, at least about 60%, at least about 70%, at least about 75%, at least about 80%, at least about 85%, at least about 90%, at least about 95%, at least about 97, at least about 99%, at least about 99.5%, or at least about 99.8%. The device may also have, in some cases, an average pore size of at least about 100 micrometers, at least about 200 micrometers, at least about 300 micrometers, at least about 400 micrometers, at least about 500 micrometers, at least about 600 micrometers, at least about 700 micrometers, at least about 800 micrometers, at least about 900 micrometers, or at least about 1 mm, and/or an average pore size of no more than about 1.5 mm, no more than about 1.4 mm, no more than about 1.3 mm, no more than about 1.2 mm, no more than about 1.1 mm, no more than about 1 mm, no more than about 900 micrometers, no more than about 800 micrometers, no more than about 700 micrometers, no more than about 600 micrometers, or no more than about 500 micrometers, etc. However, in other embodiments, further manipulation may be needed to cause the device to adopt a 3-dimensional structure, e.g., one with properties such as is discussed herein. For example, after removal of the sacrificial material, the device may need to be rolled, curled, folded, creased, etc., or otherwise manipulated to form the 3-dimensional structure. Such manipulations can be done using any suitable technique, e.g., manually, or using a machine. In some cases, the device, after insertion into matter, is able to expand, unroll, uncurl, etc., at least partially, e.g., due to the shape or structure of the device. For example, a mesh device may be able to expand after leaving the syringe.

Other materials may be also added to the device, e.g., before or after it forms a 3-dimensional structure, for example, to help stabilize the structure, to add additional agents to enhance its biocompatibility (e.g., growth hormones, extracellular matrix protein, Matrigel™ etc.), to cause it to form a suitable 3-dimension structure, to control pore sizes, etc. Non-limiting examples of such materials have been previously discussed above, and include other polymers, growth hormones, extracellular matrix protein, specific metabolites or nutrients, additional device materials, or the like. Many such growth hormones are commercially available, and may be readily selected by those of ordinary skill in the art based on the specific type of cell or tissue used or desired. Similarly, non-limiting examples of extracellular matrix proteins include gelatin, laminin, fibronectin, heparan sulfate, proteoglycans, entactin, hyaluronic acid, collagen, elastin, chondroitin sulfate, keratan sulfate, Matrigel™, or the like. Many such extracellular matrix proteins are available commercially, and also can be readily identified by those of ordinary skill in the art based on the specific type of cell or tissue used or desired.

In addition, the device can be interfaced in some embodiments with one or more electronics, e.g., an external electrical device such as a computer or a transmitter (for instance, a radio transmitter, a wireless transmitter, etc.). As mentioned, an external electrical device may be connected via one or more electrical contacts in the second portion, for example. In some cases, electrical testing of the device may be performed, e.g., before or after introduction into a living or non-living subject. For instance, one or more of the metal leads may be connected to an external electrical device, e.g., to electrically interrogate or otherwise determine the electronic state or one or more of the nanoscale wires and/or microscale wires within the device. Such determinations may be performed quantitatively and/or qualitatively, depending on the application, and can involve all, or only a subset, of the electrical elements contained within the device, e.g., as discussed herein.

The following documents are incorporated herein by reference in their entireties: U.S. Pat. No. 7,211,464, issued May 1, 2007, entitled “Doped Elongated Semiconductors, Growing Such Semiconductors, Devices Including Such Semiconductors, and Fabricating Such Devices,” by Lieber, et al.; U.S. Pat. No. 7,301,199, issued Nov. 27, 2007, entitled “Nanoscale Wires and Related Devices,” by Lieber, et al.; U.S. patent application Ser. No. 10/588,833, filed Aug. 9, 2006, entitled “Nanostructures Containing Metal-Semiconductor Compounds,” by Lieber, et al., published as U.S. Patent Application Publication No. 2009/0004852 on Jan. 1, 2009; U.S. patent application Ser. No. 10/995,075, filed Nov. 22, 2004, entitled “Nanoscale Arrays, Robust Nanostructures, and Related Devices,” by Whang, et al., published as 2005/0253137 on Nov. 17, 2005; U.S. patent application Ser. No. 11/629,722, filed Dec. 15, 2006, entitled “Nanosensors,” by Wang, et al., published as U.S. Patent Application Publication No. 2007/0264623 on Nov. 15, 2007; International Patent Application No. PCT/US2007/008540, filed Apr. 6, 2007, entitled “Nanoscale Wire Methods and Devices,” by Lieber et al., published as WO 2007/145701 on Dec. 21, 2007; U.S. patent application Ser. No. 12/308,207, filed Dec. 9, 2008, entitled “Nanosensors and Related Technologies,” by Lieber, et al.; U.S. Pat. No. 8,232,584, issued Jul. 31, 2012, entitled “Nanoscale Sensors,” by Lieber, et al.; U.S. patent application Ser. No. 12/312,740, filed May 22, 2009, entitled “High-Sensitivity Nanoscale Wire Sensors,” by Lieber, et al., published as U.S. Patent Application Publication No. 2010/0152057 on Jun. 17, 2010; International Patent Application No. PCT/US2010/050199, filed Sep. 24, 2010, entitled “Bent Nanowires and Related Probing of Species,” by Tian, et al., published as WO 2011/038228 on Mar. 31, 2011; U.S. patent application Ser. No. 14/018,075, filed Sep. 4, 2013, entitled “Methods And Systems For Scaffolds Comprising Nanoelectronic Components,” by Lieber, et al.; and Int. Patent Application Serial No. PCT/US2013/055910, filed Aug. 19, 2013, entitled “Nanoscale Wire Probes,” by Lieber, et al.

In addition, U.S. patent application Ser. No. 14/018,075, filed Sep. 4, 2014, entitled “Methods And Systems For Scaffolds Comprising Nanoelectronic Components,” by Lieber, et al., published as U.S. Patent Application Publication No. 2014/0073063 on Mar. 13, 2014; U.S. patent application Ser. No. 14/018,082, filed Sep. 4, 2013, entitled “Scaffolds Comprising Nanoelectronic Components For Cells, Tissues, And Other Applications,” by Lieber, et al., published as U.S. Patent Application Publication No. 2014/0074253 on Mar. 13, 2014; International Patent Application No. PCT/US14/32743, filed Apr. 2, 2014, entitled “Three-Dimensional Networks Comprising Nanoelectronics,” by Lieber, et al.; and U.S. Provisional Patent Application Ser. No. 61/911,294, filed Dec. 3, 2013, entitled “Nanoscale Wire Probes for the Brain and other Applications,” by Lieber, et al. are each incorporated herein by reference in its entirety.

Furthermore, U.S. Provisional Patent Application Ser. No. 61/975,601, filed Apr. 4, 2014, entitled “Systems and Methods for Injectable Devices”; and International Patent Application No. PCT/US15/24252, filed Apr. 3, 2015, entitled “Systems and Methods for Injectable Devices” are each incorporated herein by reference in its entirety. Also incorporated herein by reference in their entireties are U.S. Provisional Patent Application Ser. No. 62/201,006, filed Aug. 4, 2015, entitled “Syringe Injectable Electronics: Precise Targeted Delivery with Quantitative Input/Output,” by Lieber, et al.; and U.S. Provisional Patent Application Ser. No. 62/209,255, filed Aug. 24, 2015, entitled “Techniques and Systems for Injection and/or Connection of Electrical Devices,” by Lieber, et al.

In addition, U.S. Provisional Patent Application Ser. No. 62/505,562, filed May 12, 2017, entitled “Interfaces for Syringe-Injectable Electronics,” by Lieber, et al., is incorporated herein by reference in its entirety.

The following examples are intended to illustrate certain embodiments of the present invention, but do not exemplify the full scope of the invention.

### Example 1

Syringe-injectable mesh electronics is a promising platform for in vivo brain mapping due to its extreme flexibility, nano- to micro-scale features, and macroporous structure. These features may prevent chronic immune response and allow tracking of the same single neurons on at least a year time-scale, and thus overcome limitations of traditional neural probes. This example presents a mesh electronics design with plug-and play I/O interfacing strategy that is rapid, scalable, and user-friendly.

Mesh electronics incorporating a foldable I/O pad design were fabricated on Ni-coated Si wafers. Meshes were then fully released in Ni etchant, rinsed, and loaded in saline solution into a glass capillary tube. A stereotaxic stage and syringe pump provided spatially targeted, controlled injection of meshes into saline solution, hydrogel, and mouse (C57BL/6) brains. The I/O pads were then spread onto dicing tape, the tape edge was cut close to the pad edge, and then inserted into a custom “clamp-connect” printed circuit board (PCB). The PCB interface had either flexible flat cable (FFC) or Omnetics (A79024-001) output connectors, which were interfaced to voltage amplifiers (Intan RHD 2132) or current amplifiers (Stanford Research Systems SIM 918) for use with meshes containing standard metal electrodes or nanowire field-effect transistors, respectively. For mouse recordings, the PCB was mounted onto the skull with dental cement.

Injections of mesh electronic probes (FIG. 1, showing a schematic of a mesh electronic probe as fabricated on a silicon wafer) into hydrogel resulted in mesh injection at flow rates of 5 to 15 ml/hr and injection volumes of less than 50 microliters per 4 mm of injected mesh length. In particular, FIG. 1A shows a schematic diagram of the device, with inserts showing platinum electrodes (left), a joining region (center), and a plurality of contacts (right). FIG. 1B shows corresponding optical microscopy images.

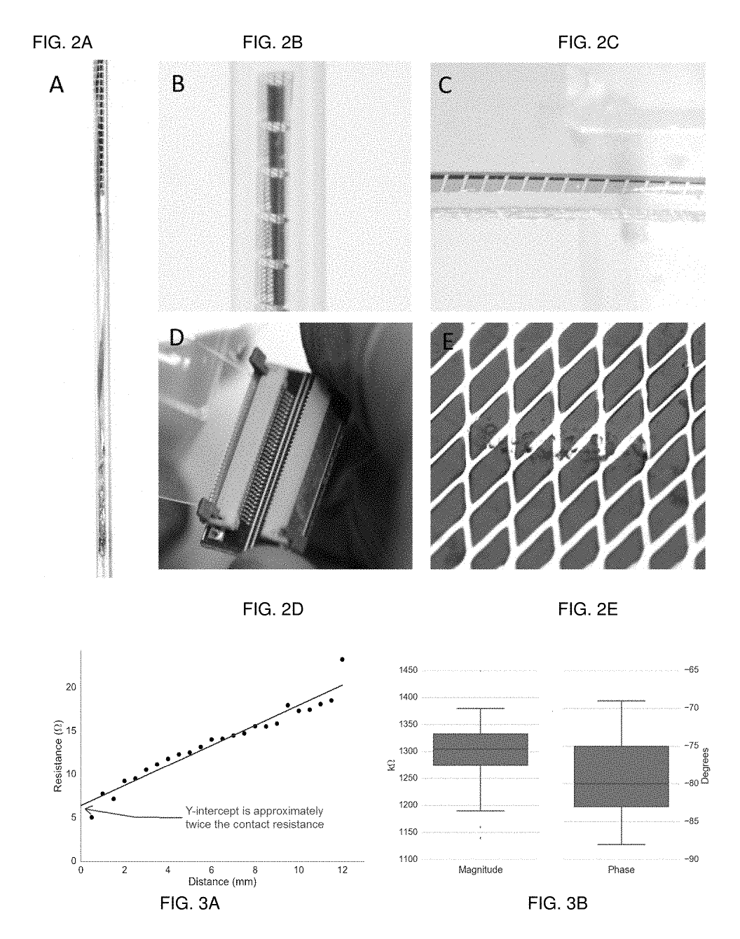

The I/O pads successfully rolled-up and passed fully through the capillary tube despite their length being larger than the tube diameter (FIG. 2A, which is a photograph of mesh I/O pads folded inside a 400 micrometer (inner diameter) capillary tube during injection). 100% of 32 recording channels were successfully connected using the plug-and-play clamp-connect PCB, typically requiring only 5 to 10 min (FIG. 2D, which is a photograph of mesh I/O pads being inserted into a clamp-connect PCB). FIG. 2B shows rolled-up contacts within the tube. The contacts were spread onto dicing tape following injection and trimmed (FIG. 2C). FIG. 2E shows indentations left on the contacts after removing the clamp-connect PCB.

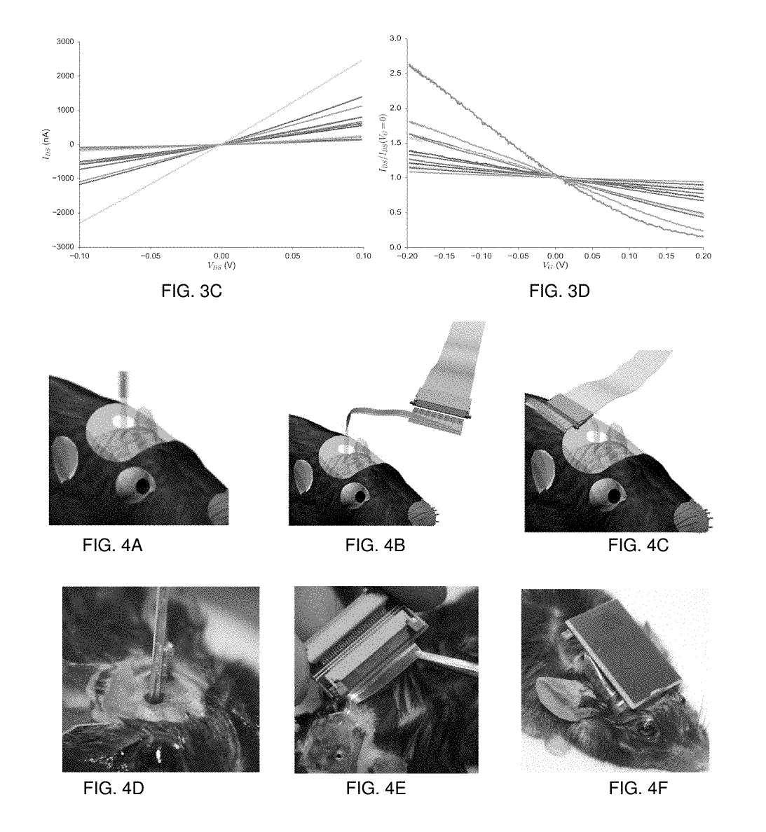

FIG. 3A shows plot of resistance vs. distance measured through a clamp-connected mesh. The y-intercept is approximately twice the contact resistance, yielding a contact resistance of about 3 Ohms. FIG. 3B shows box plots of impedance magnitude and phase for 32 platinum electrodes (20 micrometer diameter), measured at 1 kHz in 1×PBS. FIG. 3C shows I-V curves for 12 clamp-connected NW FET devices, while FIG. 3D shows W-G plots for 12 clamp-connected NW FET devices.

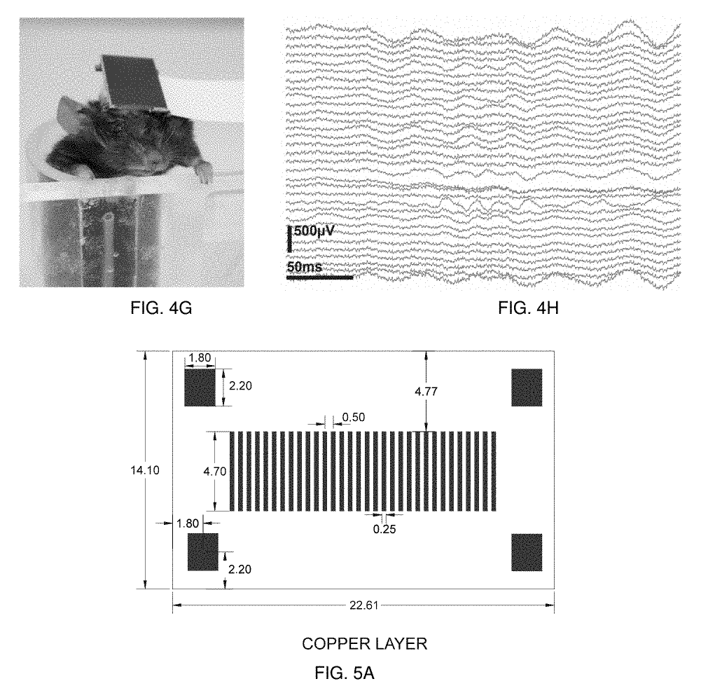

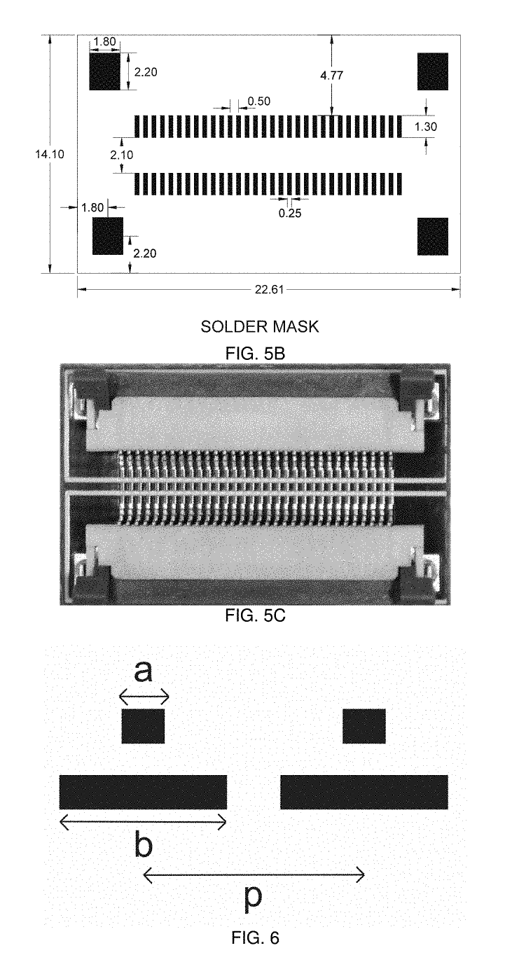

As mentioned, four-point probe measurements indicated contact resistances of only 3 ohms. The same procedure applied to mesh electronics injected into a mouse brain in vivo also resulted in 100% I/O connection yield within minutes. The PCB was easily mounted into a compact headstage and cemented in place for chronic studies (FIG. 4G, which is a photograph of a mouse during an awake-restrained recording session, highlighting how the PCB yields a compact and convenient headstage for chronic studies). Acute recordings successfully measured local field potential (LFP) from all 32 recording electrodes (FIG. 4H). FIG. 4H shows a 32-channel multiplexed local field potential (LFP) recordings from a clamp-connected mesh injected into a mouse brain. The data were recorded immediately after implantation at a 20 kHz sampling rate with a 60 Hz notch filter applied at the time of acquisition. FIG. 5 illustrates the design of a printed circuit board used in these experiments.

This example demonstrates a mesh electronics probe design with user-friendly plug-and-play I/O interfacing. This scheme was compatible with syringe-injection, and retained key features of mesh electronics responsible for seamless 3D integration in vivo. Significantly, the clamp connection required the same amount of time regardless of the number of channels being interfaced. This work allows mesh electronics with increasing channel count and sophistication, and substantially lowers barriers to use.

### Example 2

Syringe-injectable mesh electronics is a promising technology for in vivo neuroscience studies due to its macroporous structure, nano- to micro-scale features, and extreme flexibility. These unique features may prevent chronic immune response and allow tracking of the same individual neurons, unlike conventional neural probes. These same properties, however, make input/output (I/O) connection challenging, and work to-date has required materials and methods uncommon in the life sciences community. This example present as new syringe-injectable mesh electronics design with I/O interfacing that is rapid, scalable, and user-friendly to investigators in general. Data from injections into a brain-mimicking hydrogel show the mesh electronics can be delivered through syringe with precise targeting ability and microliter-scale injection volumes. Electrical characterization of mesh electronics containing Pt electrodes and silicon nanowire field-effect transistors (NW-FETs) demonstrates the ability to interface with arbitrary devices with a contact resistance of only 3 Ohm. Local field potential (LFP) recordings from an in vivo mouse injection that required only minutes for I/O connection are shown, which produced a compact, convenient head-stage compatible with chronic studies. These results expand the applicability of syringe-injectable electronics and substantially lower barriers to use for new investigators, opening the door for increasingly sophisticated and multifunctional mesh electronics in a growing array of basic and translational studies.

Syringe-injectable mesh electronics has made it possible to seamlessly innervate natural and synthetic materials with sensing and actuating electronics. Mesh electronics holds special promise for in vivo neural interfacing, where its formation of a seamless three-dimensional (3D) nanoelectronic/cellular interface with the surrounding nervous tissue represents a breakthrough approach for long-term chronic brain mapping at the level of neurons, circuits, and networks. Such electronics may prevent chronic immune response and allow tracking of the same individual neurons, thus overcoming the inflammation, gliosis, and signal inconsistency that limit traditional neural probes. Several unique features of mesh electronics account for this success: mesh electronics are delivered minimally invasively by injection through a syringe, reducing the size of the “kill zone” surrounding the implantation site; its approximately 90% open-space structure allows interpenetration by neurons and axons and the free-flow of the extracellular medium, and thus provides excellent electrochemical coupling between the mesh and the surrounding tissue; or mesh electronics possess extreme flexibility that matches the stiffness of brain tissue, preventing micro-motion and shear forces that may cause neuron signal loss and inflammation.

This example illustrates a new syringe-injectable electronics design which combines the in vivo advantages of ultra-flexible mesh electronics with the convenience of a rigid I/O region (FIG. 1A). The mesh device region maintains the macroporous, ultra-flexible mesh structure shown to promote a “neurophilic” response in vivo with the capability for long-term neuronal tracking. Pt electrodes embedded in the mesh record electrophysiological signals (FIG. 1A, i). The mesh device region tapers to a solid stem that routes high-density interconnects (FIG. 1A, ii) from the recording electrodes to I/O pads (FIG. 1A, iii). Optical microscope images confirm these design features (FIG. 1B, i-iii). The stem is designed to fit without folding inside the injecting needle (e.g. the 300 micrometer wide stem shown here was designed for injection through a 400 micrometer inner diameter capillary tube). The I/O pads, however, must be larger if to be electrically interfaced by hand. The I/O pads were made of conducting meshes with the bottom side passivated by polymer. The rhomboid lattice structure minimizes bending stiffness in the transverse direction (normal to the mesh and capillary tube axes) while maximizing bending stiffness in the longitudinal direction (parallel to the mesh and capillary tube axes). I/O pads were designed in this way to roll-up within the confined volume of the capillary tube during loading but then unfold to their full size once injected, enabling the use of I/O pads significantly larger than the diameter of the capillary tube. The solid polymer stem was robust enough to be positioned with tweezers, and its stiffness precisely maintains the relative position of each I/O pad. The I/O pad pitch (0.5 mm) and mesh lattice size (50×50 micrometer) were selected to match the conductor pitch and pin size of a zero insertion force (ZIF) connector mounted on a custom printed circuit board (PCB) (FIG. 5). The mesh electronics were fabricated on silicon wafer substrates in an entirely photolithography-based process using methods described below.

FIG. 1A shows an overview. FIG. 1A shows a schematic of syringe-injectable mesh electronics, with the ultra-flexible mesh device region at left tapering into the rigid stem and I/O regions at right. Insets provide magnified views of (i) 20 micrometer diameter Pt recording electrodes embedded in the mesh device region, (ii) the solid SU-8 stem containing an independent metal interconnect for each electrode, and (iii) the I/O pads, each of which is larger than the 400 micrometer inner diameter of the injecting needle but is foldable due to its mesh structure. The pads shown here were 0.8×0.4 mm, although pads as large as 2×0.4 mm have been injected successfully. FIG. 1B shows bright-field optical microscopy images stitched together to display syringe-injectable mesh electronics with I/O, with insets (i), (ii), and (iii) mapping to those in FIG. 1A.

### Example 3

The smallest I/O pads used here that were addressable by hand and unaided eye were approximately 0.5 to 1 mm in both dimensions. To be compatible with a 0.5 mm pitch ZIF connector and injection through a 0.4 mm inner diameter capillary tube, a normal I/O pad would have a maximum length of 0.5 mm and a width smaller than 0.4 mm (to allow passage of metal interconnects for other channels)—a challenging size for manual alignment. In these examples, alignment in the transverse direction (normal to the mesh axis) was attained by using foldable I/O pads which can be 0.8 mm or longer, as described above. Pads of this length were identifiable by naked eye and long enough to be inserted into a ZIF connector to allow for some error between insertion depth and location of the pins. Alignment in the longitudinal direction (parallel to mesh axis) was achieved by appropriate selection of pad width and spacing. For a ZIF connector with pin width a of 100 micrometers and pitch p of 500 micrometers, the mesh I/O pads had a pitch p also of 500 micrometers and pad width b=p−a, or 400 micrometers (FIG. 6). With these selections the mesh I/O pads could be inserted blindly into the ZIF connector with nearly 100% odds of 1:1 electrical interfacing (i.e. no shorted or open channels).

To evaluate the mesh electronics and the I/O interfacing scheme, mesh electronics were injected into brain-mimicking 0.5% agarose hydrogel, which has mechanical properties similar to those of brain tissue. Mesh electronics were loaded by syringe into a glass capillary tube with an inner diameter of 400 micrometers (FIG. 2A). The I/O pads folded to fit within the confined space of the capillary tube, but maintained their order and spacing due to the rigid stem and longitudinal SU-8 polymer ribbons connecting each pad (FIG. 2B). Each mesh probe was injected using the field of view (FoV) method, in which the capillary tube was retracted at the same rate with which the mesh is injected, enabling precise spatial targeting without the mesh crumpling. Typical flow rates for injection were 5-50 ml/hr and injection volumes were less than 50 microliters per injected mesh length of approximately 4 mm. After ejection and positioning of the I/O pads onto a clamping substrate (e.g., using semiconductor dicing tape) and trimming the substrate to the pad edges (see below for details), the I/O pads retained their alignment, order, and relative positions along the stem (FIG. 2C). The I/O pads were inserted and clamped into a PCB-mounted ZIF connector (FIG. 2D). Subsequent removal and microscope imaging confirmed that each ZIF connector pin clamped 1:1 onto an I/O pad with 100% yield, discernable by the indentation left behind (FIG. 2E).

Analysis of these results presents several points. First, these mesh electronics could be injected with the same capillary tube diameter, flow rates, and injection volumes, using an FoV method. Since the mesh structure injected into the brain was nearly unchanged, this indicates no compromise of in vivo chronic recording ability resulting from the new design's additional functionality. Second, anchoring the I/O pads to a stiff polymer stem avoids the tangling and random pad placement. The rigid stem imposes deterministic ordering and linear spacing of I/O pads for compatibility with a connector. The stem also possesses the practical advantage of being more robust than a macroporous mesh structure, making it amenable to facile manipulation, e.g., placement with tweezers after ejection onto a clamping substrate. Third, the mesh electronics and accompanying clamp-connect scheme worked as designed. The foldable I/O pads were long enough to be manually aligned to the edge of a clamping substrate and inserted into a ZIF connector to a depth matching that of the connector pins. The I/O pad width and pitch, designed optimally to the pin width and pitch of the ZIF connector, achieved alignment in the other dimension relying only on manual, blind insertion. The procedure was found to be rapid and had advantageous scaling properties. The entire post-injection positioning/clamping procedure usually took only 5-10 min, and was constant regardless of channel count.

FIG. 2 shows injection of mesh electronics into hydrogel and clamp-connect I/O interfacing. FIG. 2A is a photograph of mesh electronics loaded inside a 400 micrometer inner diameter capillary tube. The I/O pads are visible near the top of the image, while the mesh device region is at bottom. FIG. 2B is a photograph showing a magnified view of the I/O pads while loaded inside the capillary tube. The pads rolled-up to fit inside the constrained volume of the tube. FIG. 2C is a photograph of the I/O pads after injecting the mesh device region into hydrogel and ejecting the I/O region onto dicing tape. The dicing tape has been trimmed to the I/O pad edges with scissors. FIG. 2D is a photograph of the I/O pads being inserted into the PCB-mounted ZIF connector. FIG. 2E is an optical microscope image showing the indentation left on a mesh I/O pad after clamping/unclamping by a PCB-mounted ZIF connector. The unit cell of the I/O pad mesh must be smaller than the diameter of the ZIF connector pins to guarantee a good contact is made. If the unit cell is made too small, however, the pad will become too stiff and must be made shorter to remain injectable.

### Example 4

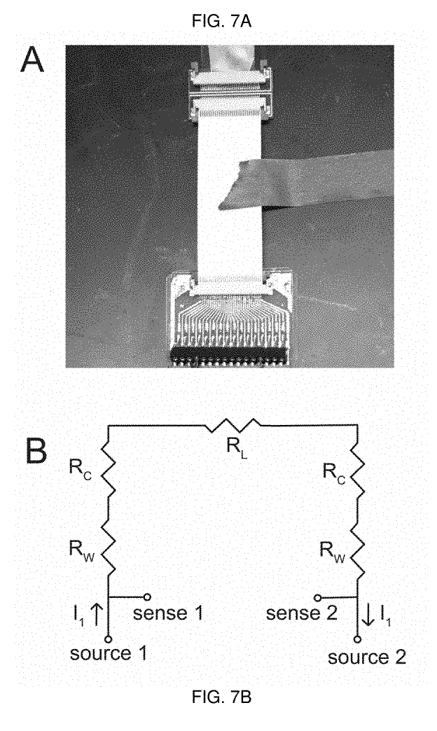

This example investigated the electrical performance of mesh electronics and the clamp-connect interfacing scheme described above through several electrical characterization experiments. To measure the contact resistance between the ZIF connector pins and the mesh I/O pads, a large area (1.5 cm×1.5 cm) mesh I/O pad was fabricated and clamped with a ZIF connector (see below for details) using the same clamp-connect protocol (FIG. 7A). Four-point probe measurements of resistance vs. distance measured from various ZIF connector channels produced data with a strong linear dependence (r²=0.94; FIG. 3A). The y-intercept represents the fixed component of the resistance, predominantly attributable to two series pad-to-pin contact resistances (FIG. 7B), yielding a contact resistance R_{c} of ca. 3 ohms.

This example also electrically characterized the device performance of the mesh electronics by measuring the 1 kHz interfacial impedance of 32 Pt electrodes in a ZIF-clamped mesh after injection into 1×phosphate buffered saline (PBS) (see below for details). The mean impedance magnitude was ca. 1.3 megohms with a mean phase of −80° (FIG. 3B), as expected for a nearly perfectly polarizable electrode. This impedance is near the theoretical purely capacitive interfacial impedance for a 20 micrometer diameter Pt electrode of ca. 930 kilohm, demonstrating that this method can successfully interface to metal recording electrodes.

To explore the generality of the above I/O strategy, this example studied mesh electronics incorporating 12 silicon nanowire field-effect transistors (NW-FETs) after injection into 1×PBS and clamp-connection (see below for details). Current-voltage (I-V) sweeps with grounded water gate measured linear output characteristics for all 12 devices (FIG. 3C), indicating ohmic contacts to the NW-FETs and the mesh electronics probe over the entire sweep range of +/−100 mV. Transfer characteristics measured with V_{DS}=100 mV and V_{G} swept over +/−200 mV showed a p-type gate response for all 12 NW-FETs (FIG. 3D). These and the above electrical characterization results highlighted the performance and generality of this I/O strategy. The small contact resistance of 3 ohm minimizes series resistance which contributes power loss and reduces transconductance in field-effect transistors (FETs), making the approach suitable for low noise/power electronics and ultra-sensitive conductance-based sensing devices.

FIG. 3 shows electrical characterization of clamp-connected mesh electronics. FIG. 3A shows four-point probe measurements of resistance vs. distance for a ZIF-clamped, large-area mesh I/O pad. The y-intercept is approximately twice the contact resistance R_{c}, yielding R_{c}˜3 ohms. FIG. 3B shows a box plot of interfacial impedance magnitude and phase at 1 kHz for 32 Pt electrodes in a ZIF-clamped mesh. All electrodes are 20 micrometers diameter and characterized while immersed in 1×PBS. FIG. 3C shows I-V curves for 12 NW-FETs in a ZIF-clamped mesh injected into 1×PBS. FIG. 3D shows water gate responses for the 12 NW-FETs shown in FIG. 3C. All devices showed a p-type response to voltages applied at a Au water gate electrode.

### Example 5

This example uses mesh electronics to in vivo live mouse brain recording. A schematic (FIG. 4A) provides an overview of the procedure (see below for details). First, mesh electronics were injected using the FoV method into the right cerebral hemisphere (FIG. 4A). Second, the I/O pads were ejected onto a clamping substrate which had been cemented in-place adjacent to the craniotomy prior to injection. The substrate was then cut with scissors to the pad edges and inserted into the ZIF connector (FIG. 4B). The entire I/O pad positioning and clamping procedure took only 5-10 min. Last, folded the PCB was folded onto the back of the mouse's skull and fixed it in place with dental cement (FIG. 4C). Photographs of the procedure (FIGS. 4D-4F) mapping to the schematic emphasize several practical points. The ability to fix the clamping substrate to the skull prior to injection is a significant advantage (FIG. 4D). After injection of the mesh into the brain and ejection of the pads onto the substrate, this arrangement minimizes relative motion between the I/O region and mesh device region, which could cause probe movement within the brain or strain that can break the mesh interconnects. This was not possible with previous interfacing methods because they required a firm, flat surface for I/O bonding. The substrate being fixed to the skull also eases pad alignment and connector insertion because the mouse can be lifted and rotated as is convenient without concern for breaking the mesh interconnects (FIG. 4E). A photograph of the PCB cemented in place highlights the convenient head-stage that results at the end of the procedure (FIG. 4F). The skull, interconnects, and ZIF connector interfacing to the I/O pads are passivated and fixed by dental cement. The ZIF connector on the opposite side of the PCB is accessible for an FFC to be inserted during recording sessions (FIG. 4G). A recording session approximately 1 hour after injection successfully recorded local field potential (LFP) from 32/32 electrode channels (FIG. 4H).

FIG. 4 shows mesh electronics for in vivo neural recording. FIGS. 4A-4C show schematics illustrating steps of the clamp-connect I/O interfacing procedure as applied to in vivo mouse brain studies. In FIG. 4A, the mesh is first injected into the brain region of interest using the FoV method followed by ejecting the I/O pads onto a clamping substrate. In FIG. 4B, the substrate is then cut with scissors to the pad edges for insertion into a PCB-mounted ZIF connector. In FIG. 4C, the PCB is cemented into place on the mouse skull, forming a convenient head-stage for acute and chronic studies. FIGS. 4D-4F illustrate photographs mapping to the schematics in FIGS. 4A-4C showing steps in the I/O interfacing procedure. The entire I/O positioning and clamping process takes only 5-10 min. FIG. 4G is a photograph of a mouse in a restrainer during a recording session. An FFC is inserted into the PCB for convenient interfacing for acute or chronic recording. FIG. 4H shows representative trace of multiplexed in vivo LFP recording from 32 Pt electrodes. Signals were sampled at 20 kHz with a 60 Hz notch filter applied at the time of acquisition and are otherwise unprocessed. Typical electrode 1 kHz impedances ranged from 1 to 1.5 megohms.

The in vivo injection of mesh electronics highlights several advantages of this design and I/O interfacing scheme. First, it reduced surgery times, with the entire procedure achievable from start to finish in only 60-90 min. Materials for interfacing included the PCB and semiconductor dicing tape. These are both commercially available and straightforward to sanitize for use in a surgical environment. The interfacing PCB functions as a convenient, durable head-stage suitable for chronic experiments. The PCB weighs only 1.54 g and has been well-tolerated by mice to-date. It also provides a convenient platform for implementing electronics for additional functionality, such as digital multiplexing, wireless communications, and signal processing. In conclusion, these examples illustrate syringe-injectable mesh electronics and an accompanying clamp-connect I/O interfacing scheme that is user-friendly, rapid, and scalable. It combines the advantages of an ultra-flexible device region that seamlessly integrates with brain tissue in vivo with the convenience of a rigid I/O stem that can be manually manipulated and aligned to a PCB-mounted ZIF connector. The design uses foldable mesh I/O pads which can be longer than the diameter of the injecting needle and I/O pad widths appropriately designed to the ZIF connector to allow 100% 1:1 connection yield with only blind insertion. Injections into brain-mimicking hydrogel validated this design and I/O scheme. Electrical characterization experiments measured a contact resistance of only 3 ohms and demonstrated functional electrical interfacing to Pt electrodes and NW-FETs, showing the generality of plug-and-play mesh electronics and the accompanying I/O strategy. The new mesh electronics design was applied to in vivo live mouse neural recordings with 100% I/O interfacing yield, successfully demonstrating LFP recording on 32 channels after an interfacing procedure that required only 5-10 min. Significantly, this may be scalable to larger channel counts, utilizes a convenient PCB head-stage that can implement new functionality, and is simple to sanitize.

### Example 6

This example illustrates various materials and methods used in the above examples.

Design of mesh electronics. In the above experiments, important parameters were: mesh width W=2.2 mm, longitudinal ribbon width w₁=20 micrometers, transverse ribbon width w₂=10 micrometers, angle between longitudinal and transverse ribbons a (alpha)=45°, longitudinal ribbon spacing L₁=333 micrometers, transverse ribbon spacing L₂=125 micrometers, metal interconnect line width wₘ=4 micrometers, and total channel count N=32. There were 19 longitudinal ribbons in a single mesh, with most carrying two interconnects with a pitch of 8 micrometers. Each transverse ribbon was V-shaped and the device region is vertically symmetric along the center longitudinal ribbon. The mesh device region was ca. 1.3 cm long, at which point it transitions to the solid stem region. The stem was 300 μmicrometers wide, extends ca. 9 mm to the first I/O pad, and carried metal interconnects of width 4 micrometers with a pitch of 9 micrometers. Parameters for the foldable mesh I/O pads were: mesh pad width is 0.5 mm, mesh pad length is 0.4 mm, pad pitch p=0.5 mm, longitudinal ribbon width is 10 micrometers, transverse ribbon width is 10 micrometers, metal conductor width is 6 micrometers, longitudinal ribbon spacing is 50 micrometers, transverse ribbon spacing is 50 micrometers, and the angle between longitudinal and transverse ribbons is 45°. Pads adjacent to a region of the stem unoccupied by interconnects extended their conductor onto the stem as far as possible, giving the largest pad a width of 0.8 mm (0.5 mm from foldable mesh pad plus 0.3 mm on the stem).

Fabrication of mesh electronics containing Pt electrodes. Important steps for fabrication of syringe-injectable mesh electronics are as follows: (i) A sacrificial layer of 100 nm Ni was thermally evaporated (Sharon Vacuum, Brockton, Mass.) onto a pre-cleaned Si wafer (n-type 0.005 ohm·cm, 600 nm thermal oxide, Nova Electronic Materials, Flower Mount, Tex.). (ii) SU-8 negative photoresist (SU-8 2000.5; MicroChem Corp., Newton, Mass.) was spin-coated onto the Si wafer at 4000 rpm for an approximate thickness of 500 nm. It was pre-baked for 1 min at 65° C. and 1 min at 95° C. before photolithography (PL) patterning (MA6 mask aligner, Karl Suss Microtec AG, Garching, Germany) at an i-line dose of 100 mJ/cm² to define the bottom polymer mesh layer. (iii) The exposed SU-8 was post-baked for 1 min at 65° C. and 1 min at 95° C. before being developed (SU-8 Developer, MicroChem Corp., Newton, Mass.) for 2 min, rinsed in isopropyl alcohol, and hard-baked at 180° C. for 1 hour. (iv) The wafer was spin-coated at 4000 rpm with LOR 3A lift-off resist (MicroChem Corp., Newton, Mass.) and baked for 5 min at 180° C., then spin-coated at 4000 rpm with Shipley 1813 positive photoresist (Microposit, The Dow Chemical Company, Marlborough, Mass.) and baked at 115° C. for 1 min. It was PL-patterned to define the metal interconnects at an h-line dose of 75 mJ/cm² followed by development (MF-CD-26, Microposit, The Dow Chemical Company, Marlborough, Mass.) for 1 min and rinsing in deionized (DI) water. (v) A 3 nm layer of Cr and 80 nm layer of Au were deposited by electron-beam evaporation (Denton Vacuum, Moorestown, N.J.). Extraneous metal was removed in a lift-off process in solvent (Remover PG, MicroChem Corp., Newton, Mass.) heated to 80° C. (vi) Steps iv and v were repeated to define 20 micrometer diameter Pt recording electrodes (3 nm Cr and 50 nm Pt). (vii) Steps ii and iii were repeated to define the top mesh polymer layer. (viii) Completed wafers were immersed in Ni etchant solution (40% FeCl₃:39% HCl:H₂O=1:1:20) to dissolve the sacrificial Ni layer and release the mesh electronic probes from the wafer. Released mesh electronics were rinsed 3 times in DI water and transferred to 1× phosphate buffered saline (PBS) before injection.

Growth of silicon nanowires. Si nanowires (NWs) were grown in a home-built chemical vapor deposition (CVD) system using the vapor-liquid-solid (VLS) process. Nanowires were catalyzed by 50 nm diameter Au nanoparticles, grown for 1 hour to reach a length of ca. 50 micrometers, and doped at a Si:B ratio of 4000:1.

Fabrication of mesh electronics containing nanowire field-effect transistors. Meshes containing silicon nanowire field-effect transistors (NW-FETs) were fabricated the same as above for steps i-iii. (iv) Nanowires (NWs) were contact printed from their growth substrates onto functionalized SiO₂. They were then spin-coated with poly(methyl methacrylate) (950 PMMA C5, MicroChem Corp., Newton, Mass.) and transferred to the device region of the mesh electronics wafer. The PMMA was dissolved in acetone, leaving behind only NWs. (v) The wafer was spin-coated at 4000 rpm with LOR 3A lift-off resist and baked for 5 min at 180° C., then spin-coated at 4000 rpm with Shipley 1813 positive photoresist and baked at 115° C. for 1 min. It was PL-patterned to define the NW contacts and metal interconnects at an h-line dose of 75 mJ/cm² followed by development in MF-CD-26 for 1 min and rinsing in DI water. NW-FETs had a channel length of 7 micrometers. (vi) The wafer was immersed in 7:1 buffered oxide etch (BOE; Transene Company, Inc., Danvers, Mass.) for 5 sec followed by 10 sec rinse in DI water. Immediately after, a 3 nm film of Cr and 80 nm film of Au were deposited by thermal evaporation. Extraneous metal was removed in a lift-off process in Remover PG heated to 80° C. (vii) Step v was repeated to mask NWs in desired locations within the mesh. Excess NWs were removed with reactive ion etching (RIE; STS MPX/LPX RIE system, SPP, Newport, United Kingdom). Masking resist was removed in Remover PG. (viii) Steps ii and iii were repeated to define the top mesh polymer layer. (ix) Completed wafers were immersed in Ni etchant solution to dissolve the sacrificial Ni layer and release the mesh electronic probes from the wafer. Released mesh electronics were rinsed 3 times in DI water and transferred to 1×PBS before injection.

Controlled injection into hydrogel and clamp-connect I/O interfacing: loading mesh electronics into capillary tubes. Needles used for injection experiments were glass capillary tubes (Drummond Scientific Co., Broomall, Pa.) with an inner diameter (I.D.) of 400 micrometers and outer diameter (O.D.) of 650 micrometers. Glass capillary tubes were inserted into a micropipette holder (Q series holder, Harvard Apparatus, Holliston, Mass.), which was connected to a 1-mL syringe (NORM-JECT®, Henke Sass Wolf, Tuttlingen, Germany) through a polyethylene intrademic catheter tubing (I.D. 1.19 mm, O.D. 1.70 mm, Becton Dickinson and Company, Franklin Lakes, N.J.). The syringe was pulled manually to draw the mesh electronics from solution into the glass capillary tube.

Preparation of Hydrogel. 0.5 g agarose (SeaPlaque® Lonza Group Ltd., Basel, Switzerland) was mixed with 100 mL DI water in a glass beaker. The beaker was covered with aluminum foil (Reynolds Wrap® Reynolds Consumer Products, Lake Forest, Ill.) to prevent evaporation and heated to boiling on a hot plate while mixed with a magnetic stir rod. Once the solution became transparent, the hot plate was switched off and the solution was allowed to cool to room temperature. The resulting hydrogel has a final mass concentration ca. 0.5% and mechanical properties similar to those of dense brain tissue.

Controlled injection of mesh electronics into hydrogel. Mesh electronics were injected by a field of view (FoV) method. Briefly, the 0.5% agarose hydrogel was poured into a cuvette to cool. A glass capillary tube loaded with mesh electronics was inserted into a micropipette holder, which was connected to a 5 mL syringe (Becton Dickinson and Company, Franklin Lakes, N.J.) via polyethylene intrademic catheter tubing (I.D. 1.19 mm, O.D. 1.70 mm). The 5 mL syringe was filled with 1×PBS and driven by a syringe pump (PHD 2000, Harvard Apparatus, Holliston, Mass.). The micropipette holder was fixed to a motorized stereotaxic stage (860A motorizer and 460A linear stage, Newport Corporation, Irvine, Calif.) for precise control of injection depth and rate. The stereotaxic stage was used to lower the end of the glass capillary tube into the hydrogel-filled cuvette to its target depth. Controlled injection was achieved by focusing an eyepiece camera (DCC1240C, Thorlabs Inc., Newton, N.J.) on the top of the mesh electronics (I/O pad region) and matching the stereotaxic retraction rate of the glass capillary tube to the rate of mesh electronics injection due to fluid flow from the syringe pump. Typical injection flow rates were 10-50 mL/h with total injection volumes less than 50 microliters per 4 mm length of injected mesh.

Clamp-connect I/O interfacing to mesh electronics. Once mesh electronics were injected to the desired depth, the glass capillary tube was repositioned using the stereotaxic stage to a clamping substrate which had been adhered to the top of the cuvette. It was found that two pieces of dicing tape (thick clear low tack roll 24353, Semiconductor Equipment Corp., Moorpark, Calif.) adhered together (adhesive sides in) worked well as a clamping substrate. The I/O pads of mesh electronics were ejected by resuming fluid flow until all I/O pads were on the tape. The I/O stem region was flipped with tweezers so the I/O pad conducting sides faced up, if necessary, and was bent at a 90° angle as near to the first I/O pad as possible. The I/O pads were rinsed by pipetting drops of DI water over them slowly; this same process was sometimes used to unfold I/O pads which occasionally did not fully extend upon ejection from the capillary tube. The I/O pads and stem were dried in place with compressed air. The dicing tape was subsequently cut with scissors to a distance of ca. 0.5 mm from the I/O pad edges; this distance was selected to align the I/O pads with the zero insertion force (ZIF; Hirose connector FH12A-32S-0.5SH(55), HIROSE Electric, Downers Grove, Ill.) connector pins when the dicing tape has been inserted as far as possible inside the connector. The trimmed tape and I/O pads were then inserted into the ZIF connector which had been mounted on a custom-made printed circuit board (PCB; Advanced Circuits, Aurora, Colo.). Once fully inserted, the ZIF connector latch was secured shut to make electrical contact with the mesh I/O pads. Successful connection was checked with an Ohmmeter or by interfacing to peripheral electronics; in case of misalignment, the tape and pads could be removed from the connector and reinserted after adjustment.

Electrical characterization of mesh electronics. Four-point probe measurements. A large-area (1.5 cm×1.5 cm) mesh I/O pad otherwise identical to those used on the mesh electronics was fabricated and clamp-connected on dicing tape using the procedures described above (FIG. 7A). The circuit resistance (FIG. 7B) was measured with a home-built four-point probe setup using a precision current source (Keithley 6220, Tektronix, Inc., Beaverton, Oreg.) and voltmeter (Analog Discovery 2, Digilent Inc., Pullman, Wash.). The lateral resistance R_{L} through the mesh I/O pad is a function of distance, while the ZIF-to-mesh contact resistance R_{C} and wire resistance R_{W} (due to a flat flexible cable, two interfacing PCBs, and pin socket wires) are fixed. A plot of resistance vs. distance, therefore, reveals a linear function with y-intercept equal to twice R_{C}+R_{W} (FIG. 3A). R_{W}=0.26 ohm and the best-fit line's y-intercept was 6.43 ohm (r²=0.94), yielding a typical contact resistance R_{C}≈3 ohm.

Electrode impedance characterization. Mesh electronics containing 32 Pt electrodes of 20 micrometer diameter were injected into 1×PBS and clamp-connected on dicing tape with the PCB-mounted ZIF connector. They were interfaced through a flat flexible cable (FFC) and home-made PCB connected to an Intan RHD 2132 amplifier system (Intan Technologies LLC, Los Angeles, Calif.). Electrode interfacial impedance was measured at 1 kHz using the Intan system's built-in electrode impedance measurement function while the 1×PBS was grounded with a Au wire.

Nanowire transistor characterization. Mesh electronics containing 12 nanowire NW-FETs were injected into 1×PBS and clamp-connected on dicing tape with the PCB-mounted ZIF connector. They were interfaced through a FFC and home-made PCB connected to a precision current amplifier (SIM918 precision current preamp and SIM900 mainframe, Stanford Research Systems, Sunnyvale, Calif.). The signals were digitized with a Digidata 1440A Digitizer (Molecular Devices, Sunnyvale, Calif.) and pCLAMP 10 data acquisition software (Molecular Devices, Sunnyvale, Calif.). Current-voltage (I-V) curves were measured by recording I_{DS} while grounding the 1×PBS with a Au wire and sweeping V_{DS} from −100 mV to +100 mV. Water gate responses were measured by applying 100 mV to V_{DS} while recording I_{DS} and sweeping the 1×PBS-immersed Au wire from −200 mV to +200 mV. Time domain signals were post-processed in Python to extract I-V and water gate curves for each device.

In Vivo injection and I/O interfacing to mesh electronics. Vertebrate animal subjects. Vertebrate animal subjects used in this study were adult (25-35 g) male C57BL/6J mice (Jackson Laboratory, Bar Harbor, Me.). All procedures performed on the vertebrate animal subjects were approved by the Animal Care and Use Committee of Harvard University. The animal care and use programs at Harvard University meet the requirements of the Federal Law (89-544 and 91-579) and National Institutes of Health (NIH) regulations and are also accredited by the American Association for Accreditation of Laboratory Animal Care (AAALAC). Animals were fed with food and water ad libitum as appropriate and were group-housed on a 12 h/12 h light/dark cycle in the Harvard University Biology Research Infrastructure (BRI).

Injection of mesh electronics into live mice brains. Briefly, all metal tools in direct contact with the mice were autoclaved for 1 h and all plastic tools in direct contact with the mice were sterilized with 70% ethanol and rinsed with sterile DI water and sterile 1×PBS before use. Mesh electronic samples were sterilized by 70% ethanol, then rinsed with sterile DI water and transferred to sterile 1×PBS. Mice were anesthetized by intraperitoneal (IP) injection of a mixture of 75 mg/kg of ketamine (Patterson Veterinary Supply Inc., Chicago, Ill.) and 1 mg/kg dexdomitor (Orion Corporation, Espoo, Finland). Mice were placed on a heating pad (Harvard Apparatus, Holliston, Mass.) set to 37° C. to maintain body temperature throughout surgery and recovery. Depth of anesthesia was monitored by pinching the mice's feet. In preparation for injection, a mouse was placed in the stereotaxic frame (Lab Standard Stereotaxic Instrument, Stoelting Co., Wood Dale, Ill.) with two ear bars and one nose clamp to fix the head in place. Hair removal lotion (Nair®, Church & Dwight, Ewing, N.J.) was applied for depilation of the mouse head and iodophor was used to sterilize the depilated scalp skin. The scalp was resected from the center axis of the skull to expose a ca. 4 mm² section of the skull. Two 1 mm diameter burr holes were formed in opposite hemispheres of the skull using a dental drill (Micromotor with On/Off Pedal 110/220, Grobet USA, Carlstadt, N.J.). The dura was carefully incised and resected, and sterile 1×PBS was swabbed on the surface of the brain to keep it moist throughout the surgery. The left burr hole was fitted with a sterilized 0-80 set screw (18-8 Stainless Steel Cup Point Set Screw; outer diameter: 0.060″ or 1.52 mm, groove diameter: 0.045″ or 1.14 mm, length: 3/16″ or 4.76 mm; McMaster-Carr Supply Company, Elmhurst, Ill.) to serve as the grounding/reference electrode. A piece of sanitized dicing tape (as prepared above) was adhered adjacent to the right burr hole with METABOND dental cement (Parkell Inc., Edgewood, N.Y.) prior to injection to serve as a clamping substrate for the I/O pads. The same injection process as described above was used for injection of mesh electronics into the live mouse brain. Typical solution volumes injected into the brain per 4 mm length of mesh were <50 microliters. I/O interfacing to in vivo mesh electronics. After injection in the brain, the capillary tube was guided to the dicing tape using the stereotaxic stage, where fluid flow was resumed to eject the mesh I/O pads. Clamp-connection to the dicing tape cemented to the mouse skull was carried out with a sterilized PCB connector using the procedure described above. The PCB was then flipped (ZIF components facing the mouse) and its end nearest to the burr hole was cemented to the exposed skull. Additional dental cement was used to secure the latch of the ZIF connector clamped to the mesh I/O pads, protect exposed areas of the mesh electronics and dicing tape, and protect the scalp, with care taken to ensure access for an FFC to be inserted in the ZIF connector on the other end of the PCB. Acute recordings were acquired approximately 1 hr after injection. Mesh electronics were interfaced via an FFC inserted into the PCB cemented to the mouse skull, which in turn connected to a home-made PCB with leads to the Intan RHD 2132 amplifier system. In vivo recording data was acquired at a 20 kHz sampling rate with a 60 Hz notch filter applied at the time of acquisition while the 0-80 set screw was used as reference. Mice were held in a Tailveiner restrainer (Braintree Scientific, LLC, Braintree, Mass.) throughout recording. Mice used only for acute recordings were euthanized via intraperitoneal injection of Euthasol at a dose of 270 mg/kg body weight.

FIG. 5 shows custom PCB for clamp-connection to mesh electronics. FIG. 5A shows a schematic of the copper routing on a custom-made PCB used to interface with plug-and-play mesh electronics (units in mm). FIG. 5B shows a schematic of the solder mask used to define component landing pads on the custom PCB (units in mm). FIG. 5C shows a photograph of the PCB after manufacturing. The PCB contains two identical ZIF connectors, with one used to clamp the mesh I/O pads and the other to interface with an FFC leading to peripheral electronics. It weighs 1.54 g.

FIG. 6 shows a geometry for I/O pad design. For a ZIF connector with pin width a and pitch p, the optimum selection of mating pad width b is b=p−a. For a larger choice of b, it becomes likely blind insertion will result in shorting adjacent channels; for smaller, it becomes likely channels will not be connected at all. When b=p−a, 1:1 interfacing occurs with nearly 100% yield.

FIG. 7 shows four-point probe measurements of contact resistance. FIG. 7A shows photograph of the experimental setup used for four-point probe contact resistance measurements. The large-area mesh I/O pad is clamped in a PCB at the top of the image. Each channel is individually addressable through an FFC interfacing to another PCB with pin socket outputs to amplifier electronics. FIG. 7B shows a circuit diagram for four-point probe measurements. The lateral resistance R_{L} through the mesh is a linear function of distance (known from the ZIF connector pin pitch of 0.5 mm). It is in series with a fixed resistance contributed by the ZIF-mesh contact resistance R_{C} plus a wire resistance R_{W} contributed by the interfacing wire, PCBs, and FFC. In a linear plot of resistance vs. distance, the y-intercept is approximately twice R_{C}+R_{W}.

### Example 7

Syringe-injectable mesh electronics raises certain challenges with electrical input/output (I/O) interfacing. The I/O pads pass fully through the needle during injection, precluding pre-bonding. The I/O pads are extremely thin and flexible, which makes it more difficult to use certain conventional semiconductor bonding techniques, such as flip-chip or wire bonding.

The “plug-and-play” interfacing method redesigns the mesh electronics probe into a leaf-shaped design with a less flexible “stem” which was able to maintain the deterministic order of the I/O pads. This allowed for them to be designed to self-align with a zero insertion force (ZIF) connector mounted on a custom printed circuit board (PCB). The interfacing takes constant time, regardless of the channel count, because the interfacing occurs during one simultaneous snap of the ZIF latch. The plug-and-play method also is compatible with surgical environments because all components can be sterilized.

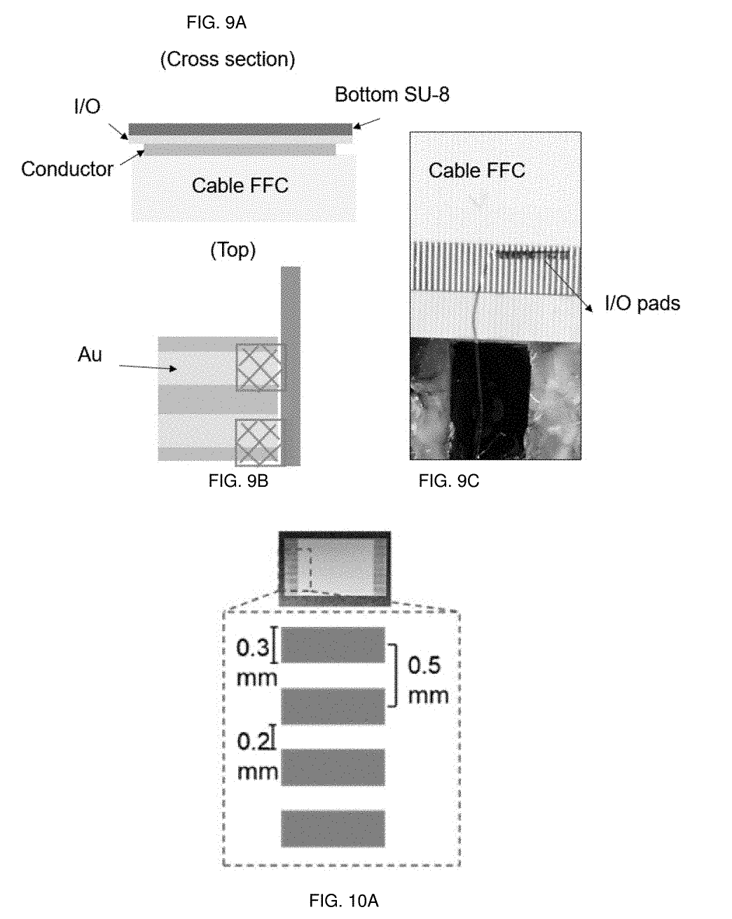

The plug-and-play design used in this example, as shown in FIGS. 9A-9C, incorporated I/O pads comprised of an electronic mesh. This design allowed for the I/O pads to roll-up during injection, making it possible to use I/O pads much larger than the injecting needle, which allows the pads to be easily interfaced by hand and naked eye to a ZIF connector. However, the mesh I/O pads could also be directly interfaced to a conductor without the use of a ZIF connector. When the pads are dried into conformal contact with the Au pads on the FFC (flat flexible cable), they form a low-resistance contact, presumably due to the extreme flexibility of the pads. The mesh design of the pads allows them to form a low-resistance contact. Importantly, the ability to directly dry the pads onto a cable greatly eases and speeds-up the interfacing time because the pads do not have to be aligned to a ZIF connector's pins.

Such a system may allow more reliable contact with compact interface can be achieved, shorter surgery time (e. g., due to easier alignment of I/O pads), and/or higher yields of contacted channels. In addition, the flexible I/O pad may allow better high-fidelity contact between I/O pads and FFC conductor.

In addition, by designing the pitch and width of the mesh I/O pads to match the conductor width and pitch on the FFC, self-alignment without shorting adjacent channels or missing other channels may be improved. The width of the pads was reduced so that they are shorter than the spacing between FFC conductors, making it difficult or impossible to short adjacent channels, e.g., due to angular misalignment. The shorter pads were still able to form a low resistance contact to the FFC when dried conformally in place.

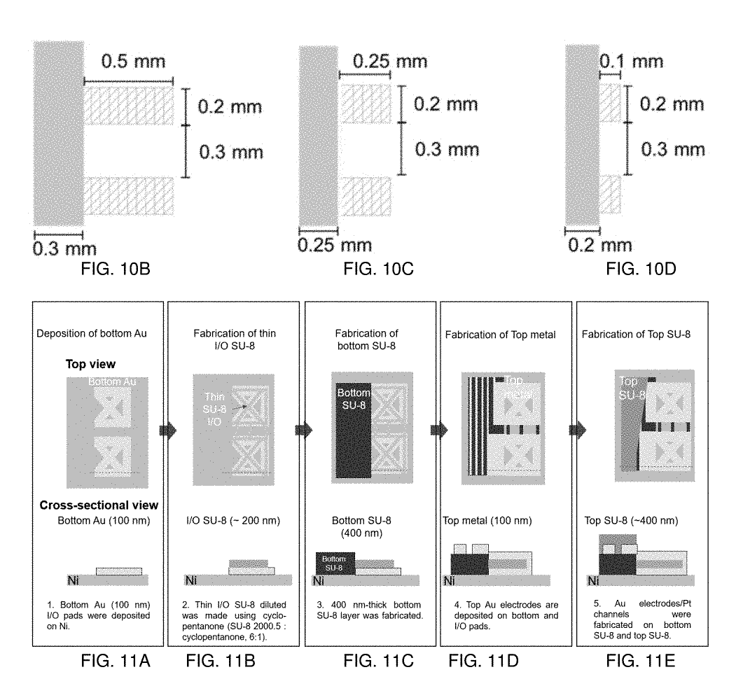

For instance, FIG. 10A shows an example FFC connector, while FIGS. 10B-10C show different designs that can be used to reduce or prevent shorting. The gap may be designed to be wider between I/O pads to avoid a short circuit. Thus, by reducing the width of I/O pads, the I/O pads cannot be short-circuited with some angles of stem.

### Example 8

This example illustrates the fabrication of double-sided I/O pads for direct contact method. If the Au (gold) conductor is only on one side of the contact, then the I/O pads must sometimes be flipped so the Au conductor is face down to mate with the FFC underneath. This takes practice and can sometimes result in breaking the mesh electronics probe. However, placing Au on the top and bottom can eliminate this problem.

This example illustrates one method of fabricating such structure. The fabrication entails (1) thermally evaporating 100 nm of Au onto a Ni sacrificial layer on a Si wafer and patterning it in a lift-off process to make the bottom Au mesh layer for the I/O pads; (2) using photolithography to pattern SU-8 into a 200-nm thick mesh support layer in the I/O pads; (3) using photolithography to pattern SU-8 into a 400-nm thick bottom SU-8 passivation layer for the mesh electronics stem and device region; (4) thermally evaporating 100 nm of Au onto the wafer and patterning it in a lift-off process to make the metal interconnects, electrodes, and top Au layer in the I/O pad region; and (5) using photolithography to pattern SU-8 into a 400-nm thick top SU-8 passivation layer that leaves open the I/O pads and electrode recording sites. The SU-8 used for the mesh I/O pads is thinned in this example using cyclopentanone to 200 nm. The thinner SU-8 is to make it easier for the thinner Au layers to sandwich and conformally coat the intervening SU-8.

Using this fabrication scheme, fabrication mesh electronics probes with double-sided I/O pads were successfully fabricated. Interfacing tests demonstrated we could electrically contact to the an FFC from both sides of the I/O pads without the need to flip them. The mesh electronics probes could be injected through a 300-micrometer inner-diameter (400-micrometer outer diameter) glass capillary needle with injection volumes as small as 20 microliters with fluid injection rates of 15 mL/h, indicating that the new I/O design does not adversely affect the injection properties.

### Example 9

This example illustrates double-sided platinum electrodes, in yet another embodiment of the invention. The steps correspond to those described in Example 8, including the deposition of platinum in step 5. In this example, the SU-8 used for the mesh I/O pads is thinned using cyclopentanone to 200 nm. The thinner SU-8 is to make it easier for the thinner Au layers to sandwich and conformally coat the intervening SU-8.

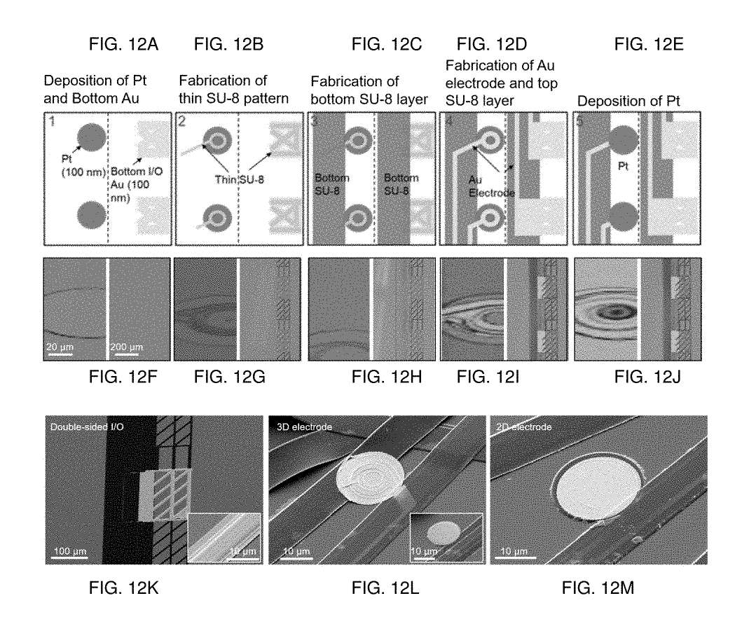

The fabrication of double-sided Pt electrodes follows the same scheme as the double-sided I/O pads described above, and can be implemented in parallel in the same mesh electronics probe. Shown in FIG. 12 are schematics (FIGS. 12A-12E) and corresponding respective optical microscopy images (FIGS. 12F-12J) of some of the key steps in the fabrication. Scanning electron microscopy images show the realized structures within a completed, released mesh (FIGS. 12K-12M).

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the invention includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A device for insertion into a subject, comprising:
a first portion comprising a plurality of electrical elements;
a second portion comprising a plurality of electrically isolated contacts; and
a joining portion electrically connecting the first portion and the second portion.

2. The device of claim 1, wherein at least some of the electrical elements are nanoscale electrical elements.

3. (canceled)

4. The device of claim 1, wherein the first portion comprises a mesh comprising the plurality of electrical elements.

5. (canceled)

6. The device of claim 1, wherein the plurality of electrical elements comprises a plurality of sensing elements.

7. The device of claim 1, wherein the plurality of electrical elements comprises a plurality of electrical elements able to apply an electrical stimulus.

8. The device of claim 1, wherein at least 50% of the electrical elements form portions of one or more electrical circuits connectable to one or more electrical circuits that are external of the device via the joining portion.

9. (canceled)

10. The device of claim 1, wherein at least some of the electrical elements are not in electrical communication with each other.

11-28. (canceled)

29. The device of claim 1, wherein at least some of the plurality of electrically isolated contacts comprises a mesh of wires.

30-44. (canceled)

45. The device of claim 1, further comprising a circuit board in electrical communication with at least some of the electrically isolated contacts.

46-53. (canceled)

54. The device of claim 45, further comprising an electrical cable in electrical communication with the circuit board.

55-80. (canceled)

81. An article, comprising:
a tube comprising the device of claim 1 contained therein.

82. (canceled)

83. The article of claim 81, wherein at least some of the plurality of electrical contacts are curled around the joining portion within the tube.

84. (canceled)

85. The article of claim 81, wherein the tube has an inner diameter of less than 500 micrometers.

86. The article of claim 81, wherein the tube is part of a syringe.

87. The device of claim 1, wherein the at least a portion of the device is positioned within a living subject.

88-98. (canceled)

99. The device of claim 87, wherein the device is immobilized relative to the subject.

100. An article, comprising:
a tube comprising a device for insertion into a subject, the device comprising a first portion comprising a plurality of electrical elements, a second portion comprising a plurality of electrical contacts, and a joining region connecting the first portion and the second portion, wherein at least some of the plurality of electrical contacts are curled around the joining region within the tube.

101-118. (canceled)

119. The article of claim 100, further comprising a circuit board physically attached to at least some of the electrically isolated contacts.

120-122. (canceled)

123. The device of claim 119, further comprising an electrical cable in electrical communication with the circuit board.

124. (canceled)

125. A method, comprising:
inserting at least a portion of a device comprising one or more electrical elements into a subject; and
attaching the device to a circuit board.

126-130. (canceled)
